(19) United States
(12) Patent Application Publication
(10) Pub. No.: US 2026/0176687 A1
(43) Pub. Date: Jun. 25, 2026

(54) METHODS AND COMPOSITIONS FOR NUCLEIC ACID SEQUENCING

(71) Applicant: 10x Genomics, Inc., Pleasanton, CA (US)

(72) Inventors: Justin COSTA, Union City, CA (US); Jeremy LACKEY, Foster City, CA (US); Trevor STOCKDALE, Walnut Creek, CA (US)

(21) Appl. No.: 19/425,819

(22) Filed: Dec. 18, 2025

Related U.S. Application Data
(60) Provisional application No. 63/736,454, filed on Dec. 19, 2024.

(51) Int. Cl. C12Q 1/6869; C12Q 1/6806; C12Q 1/6825; G01N 21/64

(52) U.S. Cl. CPC C12Q 1/6869; C12Q 1/6806; C12Q 1/6825; G01N 21/6428; G01N 21/6456; G01N 2021/6432

(57) ABSTRACT

The present disclosure relates in some aspects to methods, compositions, and kits for using nucleotides or nucleotide analogues with modifiable fluorophores to perform nucleic acid sequencing on a template nucleic acid in a biological sample, for example in a cell or a tissue sample. In some aspects, after detecting a fluorescent intensity of a nucleotide, said nucleotide is modified to become non-fluorescent, and fluorescence of other non-modified fluorescent or non-fluorescent nucleotides can be detected. In some aspects, after detecting a fluorescent intensity of a non-modified fluorescent first nucleotide, a non-fluorescent second nucleotide is modified to become fluorescent, and fluorescence of second nucleotide can be detected. Provided herein are methods that improve sensitivity and optical resolution when performing nucleic acid sequencing reaction.

### CROSS-REFERENCE TO RELATED APPLICATIONS

This application claims priority to U.S. Provisional Patent Application No. 63/736,454 filed Dec. 19, 2024, entitled “METHODS AND COMPOSITIONS FOR NUCLEIC ACID SEQUENCING,” which is herein incorporated by reference in its entirety for all purposes.

### FIELD

The present disclosure relates in some aspects to methods and compositions for sequencing nucleic acid molecules, including methods for in situ sequencing and analysis of target nucleic acids in biological samples by selectively modifying single base fluorescence.

### BACKGROUND

Nucleic acid sequencing is a versatile tool that helps scientists advance the understanding of biology and has wide-ranging applications in various fields, such as medical diagnostics, biotechnology, forensic biology, and virology. Currently, there are several sequencing methods available, including Maxam-Gilbert sequencing, Sanger (chain-termination) sequencing, and next-generation sequencing (NGS) techniques. Despite advances in nucleic acid sequencing, many challenges remain unaddressed, particularly for performing nucleic acid sequencing in cell or tissue sample.

### SUMMARY

In some aspects, the present application provides new and improved methods, compositions, and kits for profiling analytes in a sample using fluorescent nucleotides or nucleotide analogs that can be modified to a non-fluorescent state and/or using non-fluorescent nucleotides or nucleotide analogs that can be modified to a fluorescent state.

NGS sequencing-by-synthesis (SBS) is based on incorporation of a fluorescent, reversibly terminated nucleotide into an extended priming strand, where the incorporated nucleotide is complementary to a nucleotide in the template nucleic acid molecule that is being probed. In such methods, each nucleotide is labelled with a single unmodifiable fluorophore which can limit sensitivity and signal intensity. Sensitivity and optical resolution are also particularly limited when performing in situ sequencing. There is a need for methods that improve spatial resolution as optical crowding can prevent analytes from being resolved, decreasing the number of analytes detected or sequenced in a sample.

In some embodiments, provided herein are methods for sequencing a template nucleic acid molecule using a modifiable fluorescent nucleotide or nucleotide analogue, wherein a biological sample is contacted with a composition comprising a first fluorescent nucleotide analogue or modified nucleotide and a second nucleotide analogue or modified nucleotide in a fluorescent form, and wherein a cycle of a nucleic acid sequencing reaction on a template nucleic acid in the biological sample is performed.

Also provided herein, is a method of performing a cycle of a nucleic acid sequencing reaction on a template nucleic acid in the biological sample, wherein the nucleic acid sequencing reaction comprises, sequentially: a) detecting a fluorescent intensity of the second nucleotide analogue or modified nucleotide, after detecting the fluorescent intensity of the second dye, the second nucleotide analogue or modified nucleotide is modified to generate a non-fluorescent form of the second nucleotide analogue or modified nucleotide, and b) detecting a fluorescent intensity of the first fluorescent nucleotide analogue or modified nucleotide.

In some embodiments, the non-fluorescent form of the second nucleotide analogue or modified nucleotide comprises a quencher. In some embodiments, the second nucleotide analogue or modified nucleotide is modified to generate the non-fluorescent form of the second nucleotide analogue or modified nucleotide by attaching the quencher to the second nucleotide analogue or modified nucleotide. In some embodiments, the quencher is attached to the second nucleotide analogue or modified nucleotide using click chemistry. In some embodiments, the quencher is attached to the second nucleotide analogue or modified nucleotide using an azide-DBCO reaction, a tetrazine-TCO reaction, or an azide-alkyne reaction.

In some embodiments, the second nucleotide analogue or modified nucleotide comprises a pH-sensitive dye. In some embodiments, the second nucleotide analogue or modified nucleotide is modified to generate the non-fluorescent form of the second nucleotide analogue or modified nucleotide by deprotonation of the second nucleotide analogue or modified nucleotide.

In some embodiments, the second nucleotide analogue or modified nucleotide comprises a reducing agent-sensitive dye comprising a disulfide moiety, wherein the disulfide moiety is cleavable upon contact with a reducing agent, and the reducing agent-sensitive dye becomes non-fluorescent when the disulfide moiety is cleaved. In some embodiments, the reducing agent-sensitive dye is selected from the group consisting of

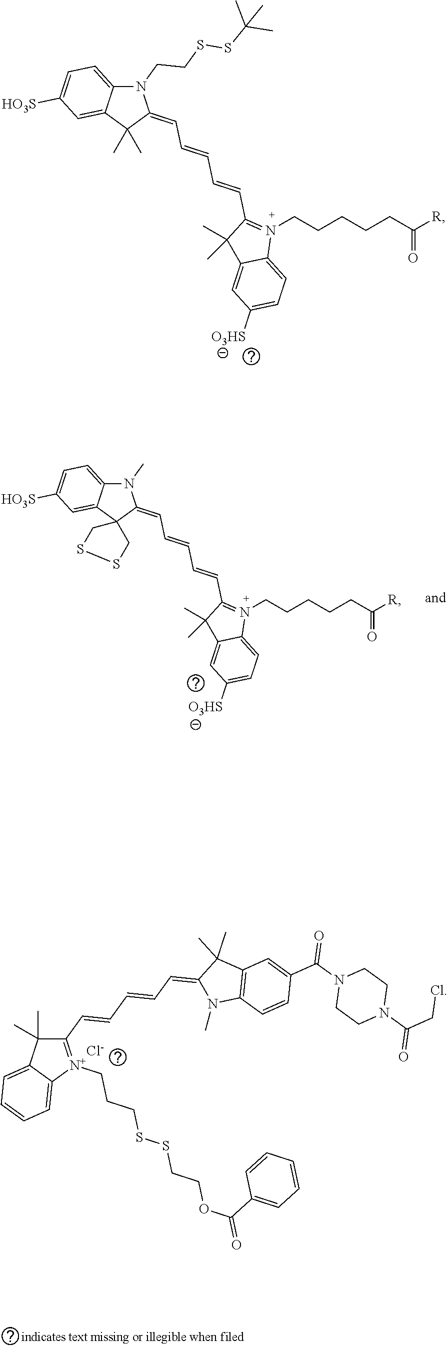

In some embodiments, provided herein are methods for sequencing a template nucleic acid molecule using a modifiable fluorescent nucleotide or nucleotide analogue, wherein a biological sample is contacted with a composition comprising a first fluorescent nucleotide analogue or modified nucleotide and a second nucleotide analogue or modified nucleotide in a non-fluorescent form, and wherein a cycle of a nucleic acid sequencing reaction on a template nucleic acid in the biological sample is performed.

Also provided herein is a method of performing a cycle of a nucleic acid sequencing reaction comprising a template nucleic acid in the biological sample, wherein the nucleic acid sequencing reaction comprises, sequentially: a) detecting a fluorescent intensity of the first fluorescent nucleotide analogue or modified nucleotide, after detecting the first fluorescent nucleotide analogue or modified nucleotide, the second nucleotide analogue or modified nucleotide is modified to generate a fluorescent form of the second nucleotide analogue or modified nucleotide, and b) detecting a fluorescent intensity of the second nucleotide analogue or modified nucleotide.

In some embodiments, the non-fluorescent form of the second nucleotide analogue or modified nucleotide comprises a quencher. In some embodiments, the second nucleotide analogue or modified nucleotide is modified to generate a fluorescent form of the second nucleotide analogue or modified nucleotide by cleaving the quencher. In some embodiments, the non-fluorescent form of the second nucleotide analogue or modified nucleotide comprises a fluorophore, and the quencher is capable of quenching the fluorophore. In some embodiments, the non-fluorescent form of the second nucleotide analogue or modified nucleotide comprises a quencher linked to the second nucleotide analogue or modified nucleotide via a cleavable linker. In some embodiments, the non-fluorescent form of the second nucleotide analogue or modified nucleotide comprises a quencher linked to the second nucleotide analogue or modified nucleotide via a reduction-cleavable disulfide bond.

In some embodiments, the second nucleotide analogue or modified nucleotide comprises a pH-sensitive dye. In some embodiments, the second nucleotide analogue or modified nucleotide is modified to generate the fluorescent form of the second nucleotide analogue or modified nucleotide by protonation of the second nucleotide analogue or modified nucleotide. In some embodiments, the pH-sensitive fluorescent dye is a rhodamine derivative. In some embodiments, the pH-sensitive fluorescent dye has the following structure under acidic conditions:

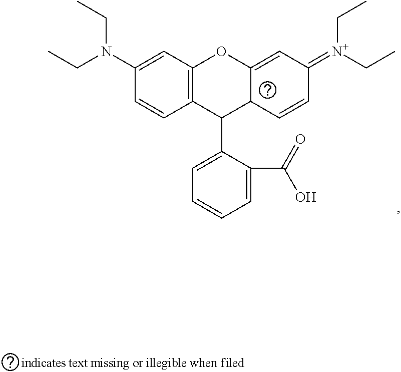 [SMILES: CCN(CC)c1ccc2c(c1)OC1=CC(=[N+](CC)CC)C=CC1C2c1ccccc1C(=O)O]

and the following structure under basic conditions:

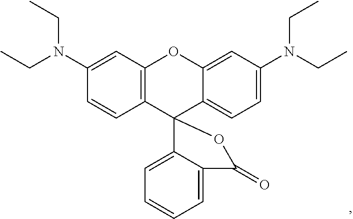 [SMILES: CCN(CC)c1ccc2c(c1)Oc1cc(N(CC)CC)ccc1C21OC(=O)c2ccccc21]

wherein the pH-sensitive dye is fluorescent in acidic conditions and non-fluorescent in basic conditions.

In some embodiments, an imaging buffer is used during the nucleic acid sequencing reaction. In some embodiments, the imaging buffer comprises an oxygen scavenger that releases a proton. In some embodiments, the pH-sensitive dye has a pKA, and in a) the imaging buffer has a pH that is above the pKA of the pH-sensitive dye, and in b) the imaging buffer has a pH that is below the pKA of the pH-sensitive dye. In some embodiments, the pH of the imaging buffer decreases during the nucleic acid sequencing reaction.

In any of the aforementioned embodiments, the first nucleotide analogue or modified nucleotide and the fluorescent form of the second nucleotide analogue or modified nucleotide emit fluorescence with distinguishable wavelengths.

In any of the aforementioned methods, the method comprises contacting the sample with a third nucleotide. In some embodiments the third nucleotide comprises a fluorescent dye that emits fluorescence detectable in a different channel than the first nucleotide analogue or modified nucleotide and the fluorescent form of the second nucleotide analogue or modified nucleotide. In some embodiments, the method further comprises contacting the sample with a fourth nucleotide. In some embodiments, the fourth nucleotide comprises a fluorescent dye that is detectable in a different channel than the fluorescent dye of the third nucleotide, the first nucleotide analogue or modified nucleotide, and the fluorescent form of the second nucleotide analogue or modified nucleotide. In some embodiments, the fourth nucleotide does not comprise a fluorescent dye.

In any of the aforementioned methods, the biological sample is contacted with a nucleotide mixture comprising the first nucleotide, the second nucleotide, the third nucleotide, and the fourth nucleotide. In some embodiments, the method comprises washing the sample to remove unbound nucleotides prior to the detecting in b) and/or prior to the detecting in c). In some embodiments, the nucleic acid sequencing reaction is a sequencing-by-synthesis reaction or a sequencing-by-binding reaction. In some embodiments, the methods comprise performing an additional cycle of a nucleic acid sequencing reaction to identify at least one additional base of the template nucleic acid. In some embodiments, the at least one additional cycle comprises at least 2, 5, 10, 20, 30, 40, or 50 additional cycles.

In any of the aforementioned embodiments, the first nucleotide, second nucleotide, third nucleotide, and fourth nucleotide comprise different nucleobases selected from the group consisting of A, T or U, C, and G. In some embodiments, the first nucleotide, second nucleotide, third nucleotide, and fourth nucleotide are reversibly terminated nucleotides. In some embodiments, the template nucleic acid comprises a DNA molecule. In some embodiments, the template nucleic acid comprises an RNA molecule. In some embodiments, the template nucleic acid comprises a target analyte nucleic acid sequence. In some embodiments, the template nucleic acid comprises a barcode sequence associated with an analyte in the biological sample.

In any of the aforementioned embodiments, prior to the nucleic acid sequencing reaction, a circularizable probe hybridizes to a target analyte in the biological sample or to a labeling agent bound to the target analyte and is ligated to form a circularized probe. In some embodiments, the method further comprises performing rolling circle amplification of the circularized probe to generate the template nucleic acid. In some embodiments, the template nucleic acid is a rolling circle amplification product (RCP). In some embodiments, the circularizable probe is a padlock probe sequence. In some embodiments, the probe sequence comprises one or more barcode region. In some embodiments, the target analyte comprises an mRNA molecule. In some embodiments, the template nucleic acid to be sequenced is attached to a solid support. In some embodiments, the solid support comprises a sequencing flow cell. In some embodiments, the template nucleic acid is a first template nucleic acid in the biological sample, and wherein the method comprises performing the cycle of the nucleic acid sequencing reaction on a second template nucleic acid in the biological sample. In some embodiments, the first template nucleic acid comprises a nucleotide complementary to the first nucleotide analogue or modified nucleotide that is identified in the cycle of the nucleic acid sequencing reaction. In some embodiments, the second template nucleic acid comprises a nucleotide complementary to the second nucleic acid analogue or modified nucleotide. In some embodiments, the first template nucleic acid and the second template nucleic acid are at optically overlapping locations in the biological sample.

In any of the aforementioned embodiments, the biological sample is a cell or tissue sample. In some embodiments, the biological sample is a fresh frozen tissue section. In some embodiments, the biological sample is a paraffin embedded formalin fixed (FFPE) tissue section. In some embodiments, the biological sample is immobilized on a surface. In some embodiments, the template nucleic acid molecule is sequenced in situ in the cell sample or tissue sample. In some embodiments, the cell sample comprises a layer of cells deposited on a surface.

Provided herein are also kits for performing a cycle of a nucleic acid sequencing reaction. In some embodiments, the kit comprises a first fluorescent nucleotide analogue or modified nucleotide, and a second nucleotide analogue or modified nucleotide in a fluorescent form. In some embodiments, the second nucleotide analogue or modified nucleotide is configured to be modifiable to generate a non-fluorescent form of the second nucleotide analogue or modified nucleotide. In some embodiments, the kit further comprises a quencher that is configured for attachment to the second nucleotide analogue or modified nucleotide. In some embodiments, the quencher is configured for attachment to the second nucleotide analogue or modified nucleotide using click chemistry. In some embodiments, the quencher is configured for attachment to the second nucleotide analogue or modified nucleotide using an azide-DBCO reaction, a tetrazine-TCO reaction, or an azide-alkyne reaction. In some embodiments, the second nucleotide analogue or modified nucleotide comprises a sulfur-containing-sensitive dye that is deactivatable by exposure to a reducing agent.

In some embodiments, a kit for performing a cycle of a nucleic acid sequencing reaction comprises a first fluorescent nucleotide analogue or modified nucleotide and a second nucleotide analogue or modified nucleotide in a non-fluorescent form. In some embodiments, the second nucleotide analogue or modified nucleotide is configured to be modifiable to generate a fluorescent form of the second nucleotide analogue or modified nucleotide. In some embodiments, the second nucleotide analogue or modified nucleotide in the non-fluorescent form comprises a cleavable quencher. In some embodiments, the second nucleotide analogue or modified nucleotide in the non-fluorescent form comprises a pH-sensitive dye that is activatable by protonation.

Further, provided herein are compositions to perform the aforementioned methods. In some embodiments, a composition comprises a first nucleotide comprising a first dye and a second nucleotide comprising a second dye, wherein the first dye comprises a quencher. In some embodiments, the quencher comprises a cleavable moiety. In some embodiments, a composition comprises a first nucleotide comprising a first dye and a second nucleotide comprising a second dye, wherein the first dye comprises a quencher-attachment moiety wherein a quencher attaches. In some embodiments, a composition comprises a first nucleotide comprising a first dye, a second nucleotide comprising a second dye, wherein the first dye is pH-sensitive, emitting fluorescence in an acidic environment and not in a basic environment. In some embodiments, a composition comprises a first nucleotide comprising a first dye, a second nucleotide comprising a second dye, wherein the first dye is reducing agent-sensitive, emitting fluorescence in a nonreducing environment and not emitting fluorescence in a reducing environment.

### BRIEF DESCRIPTION OF THE DRAWINGS

The drawings illustrate certain features and advantages of this disclosure. These embodiments are not intended to limit the scope of the appended claims in any manner.

FIG. 1 depicts an example workflow for nucleic acid sequencing comprising modifying a second nucleotide analogue or modified nucleotide by attaching a quencher to the second nucleotide analogue or modified nucleotide, thereby modifying the second nucleotide analogue or modified nucleotide from a fluorescent form to a non-fluorescent form.

FIG. 2 depicts an example workflow for nucleic acid sequencing comprising modifying a second nucleotide analogue or modified nucleotide by cleaving a quencher from the second nucleotide analogue or modified nucleotide, thereby modifying the second nucleotide analogue or modified nucleotide from a fluorescent form to a non-fluorescent form.

FIG. 3 depicts an example workflow for nucleic acid sequencing comprising modifying a second nucleotide analogue or modified nucleotide comprising a pH-sensitive dye. In this example, as the pH decreases, the pH-sensitive dye fluoresces and is “ON” (FIG. 3, right).

FIG. 4 depicts an example workflow for nucleic acid sequencing comprising modifying a second nucleotide analogue or modified nucleotide comprising a reducing agent-sensitive dye. In this example, upon exposure to a reducing agent, the reducing agent-sensitive dye is turned “OFF” (FIG. 4, right).

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Methods for sequencing nucleic acid molecules described herein use fluorescent nucleotides or nucleotide analogs that can be modified to a non-fluorescent state and/or using non-fluorescent nucleotides or nucleotide analogs that can be modified to a fluorescent state. Further, described herein are new and improved methods, compositions, and kits for NGS, including SBS and sequencing by hybridization (SBH). In some embodiments, a method provided herein comprises using modifiable fluorescent nucleotides or nucleotide analogues to perform a cycle of a nucleic acid sequencing reaction on a template nucleic acid in the biological sample. In some embodiments, the template nucleic acid molecule is sequenced in situ in the cell sample or tissue sample.

Provided herein are methods for sequencing using quenchable fluorophores. In some embodiments, the quenchable fluorophore comprises an attachment moiety for quenchers. In some embodiments, a method provided herein is a method of sequencing at least a portion of a template nucleic acid in a cell or tissue sample, the method comprising: (a) contacting a biological sample with a composition comprising a first fluorescent nucleotide analogue or modified nucleotide and a second nucleotide analogue or modified nucleotide in a fluorescent form; and (b) performing a cycle of a nucleic acid sequencing reaction on a template nucleic acid in the biological sample, wherein the nucleic acid sequencing reaction comprises, sequentially: a) detecting a fluorescent intensity of the second nucleotide analogue or modified nucleotide, wherein after detecting the fluorescent intensity of the second dye, the second nucleotide analogue or modified nucleotide is quenched by introducing (e.g., attaching) a quencher, which generates a non-fluorescent form of the second nucleotide analogue or modified nucleotide, and b) detecting a fluorescent intensity of the first fluorescent nucleotide analogue or modified nucleotide.

Provided herein are methods for sequencing using fluorophores comprising a reducing agent-sensitive dye. In some embodiments, a method provided herein is a method of sequencing at least a portion of a template nucleic acid in a cell or tissue sample, the method comprising: (a) contacting a biological sample with a composition comprising a first fluorescent nucleotide analogue or modified nucleotide and a second nucleotide analogue or modified nucleotide in a fluorescent form; and (b) performing a cycle of a nucleic acid sequencing reaction on a template nucleic acid in the biological sample, wherein the nucleic acid sequencing reaction comprises, sequentially: a) detecting a fluorescent intensity of the second nucleotide analogue or modified nucleotide, wherein after detecting the fluorescent intensity of the second dye, the second nucleotide analogue or modified nucleotide is exposed to a reducing agent, thereby cleaving a disulfide moiety associated with the reducing agent-sensitive dye and generating a non-fluorescent form of the second nucleotide analogue or modified nucleotide, and b) detecting a fluorescent intensity of the first fluorescent nucleotide analogue or modified nucleotide.

Provided herein are methods for sequencing using activatable fluorophores, wherein the fluorophore may be modified to transition from a non-fluorescent to a fluorescent state. In some embodiments, a method provided herein is a method of sequencing at least a portion of a template nucleic acid in a cell or tissue sample, the method comprising: (a) contacting a biological sample with a composition comprising a first fluorescent nucleotide analogue or modified nucleotide and a second nucleotide analogue or modified nucleotide, wherein the second nucleotide analogue or modified nucleotide is in a non-fluorescent form and comprises a quencher; and (b) performing a cycle of a nucleic acid sequencing reaction comprising on a template nucleic acid in the biological sample, wherein the nucleic acid sequencing reaction comprises, sequentially: a) detecting a fluorescent intensity of the first fluorescent nucleotide analogue or modified nucleotide, wherein after detecting the first fluorescent nucleotide analogue or modified nucleotide, the second nucleotide analogue or modified nucleotide is modified to cleave the quencher to generate a fluorescent form of the second nucleotide analogue or modified nucleotide, and b) detecting a fluorescent intensity of the second nucleotide analogue or modified nucleotide.

Provided herein are methods for sequencing using pH-sensitive fluorophores. In some embodiments, a method provided herein is a method of sequencing at least a portion of a template nucleic acid in a cell or tissue sample, the method comprising: (a) contacting a biological sample with a composition comprising a first fluorescent nucleotide analogue or modified nucleotide and a second nucleotide analogue or modified nucleotide, wherein the second nucleotide analogue or modified nucleotide is in a non-fluorescent form and in a basic environment; and (b) performing a cycle of a nucleic acid sequencing reaction comprising on a template nucleic acid in the biological sample, wherein the nucleic acid sequencing reaction comprises, sequentially: a) detecting a fluorescent intensity of the first fluorescent nucleotide analogue or modified nucleotide, wherein after detecting the first fluorescent nucleotide analogue or modified nucleotide, the second nucleotide analogue or modified nucleotide is protonated in an acidic environment, generating a fluorescent form of the second nucleotide analogue or modified nucleotide, and b) detecting a fluorescent intensity of the second nucleotide analogue or modified nucleotide. In some embodiments, a method provided herein is a method of sequencing at least a portion of a template nucleic acid in a cell or tissue sample, the method comprising: (a) contacting a biological sample with a composition comprising a first fluorescent nucleotide analogue or modified nucleotide and a second nucleotide analogue or modified nucleotide in a fluorescent form; and performing a cycle of a nucleic acid sequencing reaction on a template nucleic acid in the biological sample, wherein the nucleic acid sequencing reaction comprises, sequentially: a) detecting a fluorescent intensity of the second nucleotide analogue or modified nucleotide, wherein after detecting the fluorescent intensity of the second dye, the second nucleotide analogue or modified nucleotide is modified in a higher pH environment to generate a non-fluorescent form of the second nucleotide analogue or modified nucleotide, and b) detecting a fluorescent intensity of the first fluorescent nucleotide analogue or modified nucleotide.

Advantageously, when sequencing with nucleotides or nucleotide analogues comprising quenchable fluorophores, reducing agent-sensitive dyes, activatable fluorophores, and/or pH-sensitive fluorophores, detection sensitivity and signal intensity is improved. Modulating fluorescence throughout one or more cycles of a nucleic acid sequencing reaction allows for improved sensitivity and optical resolution. Modifiable fluorophores are described in greater detail in Section II.

The provided methods are particularly useful for nucleic acid sequencing reactions in cell or tissue samples, as in situ sequencing requires improved spatial resolution and amelioration of optical crowding to increase the number of analytes detected or sequenced in a sample. Performing nucleic acid sequencing using modifiable fluorophores is described in greater detail in Section III. Additional aspects of the methods, compositions, kits, and systems disclosed herein are described in the sections below (e.g., compositions are further described in Section VIII, systems are further described in Section VII, and kits are further described in Section IX).

In some aspects, the methods disclosed herein comprise a biological sample and preparing the biological sample prior to performing the methods. Biologicals samples and sample preparation is described in detail in Section IV. In some embodiments, preparing a sample comprises (a) preparation, (b) embedding, and/or c) staining and immunohistochemistry (IHC).

In some aspects, the methods disclosed herein comprise performing nucleic acid sequencing reaction on a template nucleic acid. In some embodiments, the template nucleic acid comprises one or more analytes. Analytes, including but not limited to endogenous analytes, are described in Section V. In some aspects, provided herein are methods and compositions for analyzing one or more products of an endogenous analyte and/or a labeling agent in a biological sample. In some instances, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed (e.g., further described in Section V).

In some aspects, the methods disclosed herein includes “cyclic array sequencing” of amplified template nucleic acid molecules. Cyclic array flow cell sequencing methods generally involve performing multiple cycles of an enzymatic reaction on an array of spatially-separated oligonucleotide features (e.g., clonally-amplified colonies of template nucleic acid fragments tethered to a support surface, e.g., a flow cell surface). Cyclic array sequencing in a flow cell is further described in Section VI.

### II. Fluorescent and Non-Fluorescent Forms of Nucleotide Analogues or Modified Nucleotides

In some embodiments, each cycle of a base-by-base sequencing reaction performed as part of the disclosed methods comprises contacting priming strands bound to template nucleic acid molecules with a composition comprising nucleotides, wherein the nucleotides comprise fluorophores. In some embodiments, for one or more cycles of the base-by-base sequencing reaction, the composition comprises one or more modifiable fluorescent nucleotides or nucleotide analogues as described herein (e.g., wherein the fluorophore or fluorescent nucleotide analogue is modifiable by attachment of a quencher, cleavage of a quencher, or uncaging fluorescence by cleaving a chemical bond). In some embodiments, the method comprises selectively modifying a nucleotide analogue or modified nucleotide from a fluorescent form to a non-fluorescent form (e.g., by attaching a quencher, or by changing the pH wherein the nucleotide analogue or modified nucleotide comprises a pH-sensitive dye). In some embodiments, the method comprises selectively modifying a nucleotide analogue or modified nucleotide from a non-fluorescent form to a fluorescent form (e.g., by cleaving a quencher, or by changing the pH wherein the nucleotide analogue or modified nucleotide comprises a pH-sensitive dye).

In some embodiments, modifying fluorophores allows for a controlled change from a fluorescent state to a non-fluorescent state or from a non-fluorescent state to a fluorescent state. For example, in some cases modifying a fluorophore by quenching, for example by attaching a quencher, decreases the fluorescence intensity. In some embodiments, fluorescence quenching is be achieved with reductive agents and/or fluorophores with quencher-ligand conjugates. Modifying fluorescence by quenching is further described below. In some embodiments, the method comprises removing a quencher from a fluorophore-quencher conjugate to modify a fluorophore's state from non-fluorescent to fluorescent. Modifying fluorescence by removing quenchers is further described below.

In some embodiments, the fluorescent state of a fluorophore is dependent on pH as fluorescent proteins or fluorophores may be pH-sensitive. For example, changes in pH throughout a cycle of a nucleic acid sequencing reaction affects the fluorescence of these proteins or fluorophores. pH-sensitive fluorophores are described in further detail below.

In some embodiments, the nucleotide or set of nucleotides comprise a modifiable fluorophore. In some embodiments, the nucleotide or set of nucleotides comprise a pteridine nucleoside. In some embodiments, each cycle of a cyclic series of base-by-base sequencing reactions comprises contacting the priming strand bound to a template nucleic acid molecule with a composition comprising a plurality of sets of nucleotides (e.g., 2, 3, or 4 sets of nucleotides). In some embodiments, each set of nucleotides comprises a same nucleobase that differs from the other sets. In some embodiments, a composition comprises sets of nucleotides, wherein each set comprises a same nucleobase (e.g., selected from A, T, U, C, and/or G) that differs from the other sets, and wherein, for each set, nucleotides are labeled with a same fluorophore that differs from the other sets.

In some instances, the modifiable fluorophore comprises a fluorophore from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 542, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

### Quenchers

In some embodiments, sequencing methods disclosed herein include modifiable nucleotides that are modifiable to add or remove a quencher moiety. Fluorescence quenching includes any process that decreases the fluorescence intensity, wherein the quenching is due to molecular interactions between the fluorophore and quencher. In some embodiments, fluorophores donate an electron to the quencher. For example, indole, tryptophan, and its derivatives are quenched by acrylamide, succinimide, dichloroacetamide, dimethylformamide, pyridinium hydrochloride, imidazolium hydrochloride, methionine, Eu3+, Ag+, and Cs+.

In some embodiments, a modifiable nucleotide is in a non-fluorescent form, and comprises a fluorophore moiety and an attached quencher moiety, wherein the attached quencher moiety may be cleaved. Upon cleavage of the quencher moiety, the modifiable nucleotide is converted (e.g., transitioned) to a fluorescent form.

In some embodiments, a modifiable nucleotide is in a fluorescent form, and comprises a fluorophore moiety and an attachment moiety (e.g., a first click-chemistry attachment moiety). Upon attachment of a quencher comprising an attachment moiety (e.g., a second click-chemistry attachment moiety), the modifiable nucleotide is converted (e.g., transitioned) to a non-fluorescent form.

In some embodiments, the quencher is attached to a fluorophore in the second nucleotide analogue or modified nucleotide to quench fluorescence and transition the fluorophore to a non-fluorescent state. In some embodiments, the second nucleotide or modified nucleotide comprises a quencher, wherein the quencher is cleaved to transition the fluorophore of the second nucleotide or modified nucleotide to a fluorescent state. Non-limiting examples of quenchers may comprise acrylamide, amines, amine anesthetics, bromate, bromobenzene, carboxy groups, cesium, chlorinated compounds, cobalt, dimethylformamide, disulfides, ethers, halogens, halogen peroxide, iodide, imidazole, histidine, indole, methylmercuric chloride, nickel, nitromethane, nitroxides, nitric oxide, olefins, oxygen, peroxides, picolinium nicotinamide, pyridine, silver, succinimide, sulfur dioxide, thallium, thiocyanate, or xenon. In some embodiments, the quencher is selected from Black Hole Quenchers (e.g., Black Hole-1, Black Hole-2, and Black Hole-3), Qxl quenchers, Iowa Black FQ, Iowa Black RQ, and IRDye QC-1. In some embodiments, the modifiable nucleotide comprises a quencher. In some embodiments, the quencher comprises a black hole quencher or a dabcyl moiety.

A quencher of a modified nucleotide or nucleotide analogue as described herein may be directly or indirectly linked to the nucleotide and corresponding fluorophore. In some embodiments, the quencher is configured for attachment to the nucleotide analogue or nucleotide using an azide-DBCO reaction, a tetrazine-TCO reaction, or an azide-alkyne reaction. In some embodiments, the quencher is attached to the modifiable nucleotide or nucleotide analogue using click chemistry.

In some embodiments, the quencher is attached via a linker. In some embodiments, a linker includes a moiety generated by click chemistry conjugation of a first click chemistry reacting group attached to a nucleotide molecule with a second click chemistry group attached to the quencher. In some instances, the nucleotide molecule and the quencher are conjugated via first and second click reactive functional groups using a click reaction. In some embodiments, the first click reactive functional group and second click reactive functional group are selected from: azido/alkynyl groups; alkynyl/azido groups; azido/dibenzocyclooctynyl (DBCO) groups; dibenzocyclooctynyl (DBCO)/azido groups; azido/cyclooctynyl groups; cyclooctynyl/azido groups; tetrazine/dienophile groups; dienophile/tetrazine groups; thiol/alkynyl groups; alkynyl/thiol groups; cyano/1,2-amino thiol groups; 1,2-amino thiol/cyano groups; nitrone/cyclooctynyl groups; cyclooctynyl/nitrone groups; or any combination thereof.

In some embodiments, the quencher is cleaved to generate a fluorescent form of the nucleotide or nucleotide analogue comprising the quencher. In some embodiments, the quencher is linked via a cleavable linker. In some embodiments, the quencher is linked via a reduction-cleavable disulfide bond.

In some instances, the cleavable linker comprises a photocleavable linker, a Pd-cleavable linker, or a reducing agent-cleavable linker such as a phosphine-cleavable linker or a disulfide linker.

In some instances, the cleavable linker includes a photocleavable linker. Any suitable photocleavable linker can be used (see, e.g., Seo et al. (2005), PNAS 102(17): 5926-5931, which is incorporated by reference herein in its entirety). In some instances, the photocleavable linker comprises a nitrobenzyl group. For instance, a photocleavable nitrobenzyl linker can be cleaved using laser irradiation (e.g., 355 nm, 10 seconds, 1.5 Wcm⁻²).

In some instances, the cleavable linker includes a Pd-cleavable linker. Any suitable Pd-cleavable linker can be used (see, e.g., Ju et al. (2006), PNAS 103(52): 19635-19640, which is incorporated by reference herein in its entirety). In some instances, the Pd-cleavable linker comprises an allyl group. For instance, a Pd-cleavable allyl linker can be cleaved using incubation with a Na₂PdCl₄/P(PhSO₃Na)₃ mixture (e.g., 30 seconds at 70° C.).

In some instances, the cleavable linker is a reducing agent-cleavable linker. Examples of reducing agents that may be used to cleave a reducible linker include Tris(2-carboxyethyl) phosphine and dithiothreitol (DTT). Examples of reducible moieties that may be included in a linker include disulfide and azidomethyl. In some instances, the cleavable linker includes a phosphine-cleavable linker. Any suitable phosphine-cleavable linker can be used (see, e.g., Guo et al. (2008), PNAS 105(27): 9145-9150, which is incorporated by reference herein in its entirety). In some instances, the phosphine-cleavable linker comprises an azido group. For instance, a phosphine-cleavable azido linker can be cleaved using incubation with a Tris(2-carboxyethyl) phosphine (TCEP) mixture (e.g., 15 minutes at 65° C.).

In some instances, the cleavable linker includes a disulfide bond. For instance, the disulfide bond can be cleaved using incubation with a reducing agent, such as beta-mercaptoethanol, TCEP, or dithiothreitol (DTT).

### Reducing Agent-Sensitive Fluorophores

In some embodiments, provided herein is a method comprising: contacting a biological sample with a composition comprising a first nucleotide comprising a first dye and a second nucleotide comprising a reducing agent-sensitive dye; and performing a cycle of a nucleic acid sequencing reaction comprising a template nucleic acid in the biological sample, wherein the nucleic acid sequencing reaction comprises, sequentially: a) detecting a fluorescent intensity of the second nucleotide, b) contacting the composition with a reducing agent, thereby converting the reducing-agent sensitive dye to a non-fluorescent form, and c) detecting a fluorescent intensity of the first nucleotide. In some embodiments, the reducing agent-sensitive fluorophore is a modified sulfo-Cy5 including a moiety that contains a disulfide bond. Without being bound by theory, the thiol generated upon disulfide cleavage can disrupt the fluorescence by conjugate addition into the resonance structure of the fluorophore. In some embodiments, the modifiable fluorophore comprises sulfur. In some embodiments, the modifiable fluorophore comprises a disulfide moiety, wherein the disulfide moiety is cleavable upon contact with a reducing agent. Fluorophores with a cleavable disulfide moiety that change the fluorescent state of the fluorophore when exposed to a reducing agent are also referred to as reducing agent-sensitive dyes. In some embodiments, the nucleotide or nucleotide analogue to be modified comprises a reducing agent-sensitive dye. In some embodiments, the reducing agent-sensitive dye becomes non-fluorescent when the disulfide moiety is cleaved. Thus, in some examples, wherein the modifiable nucleotide comprises a reducing agent-sensitive dye, the fluorophore may be quenched when exposed to a reducing agent. In some embodiments, the nucleotide or nucleotide analogue comprising a reducing agent-sensitive dye is modified with a reducing agent.

Reducing agents selected for use during a cycle of nucleic acid sequencing reaction may be substance that donates electrons to an electron recipient. Reducing agents may comprise lithium, sodium, magnesium, aluminum, hydrogen gas, chromium, cuprous, and/or chloride. In some embodiments, reducing agents comprise calcium, barium, alkali metals, formic acid, oxalic acid, and/or sulfite compounds. For example, commonly used reducing agents include lithium aluminum hydride, Red-Al, hydrogen, sodium amalgam, sodium-lead allot, zinc amalgam, diborane, sodium borohydride, ferrous compounds, stannous, sulfur dioxide, dithionates, thiosulfates, and dithiothreitol (DTT).

In some embodiments, the reducing agent-sensitive dye emits fluorescence in a nonreducing environment and does not emit fluorescence in a reducing environment. In some embodiments, the reducing agent-sensitive dye is selected from the group consisting of

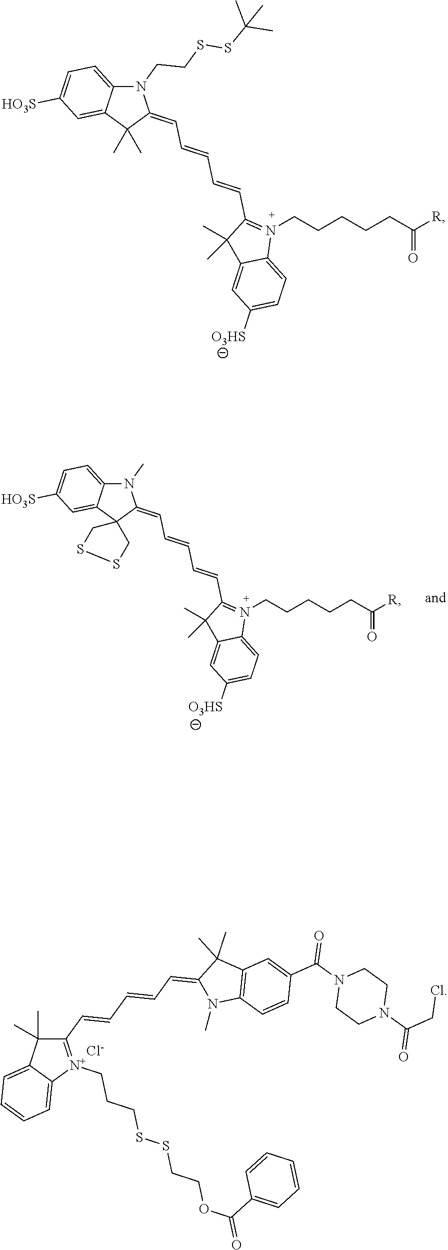

In some embodiments, the reducing agent-sensitive dye has the structure:

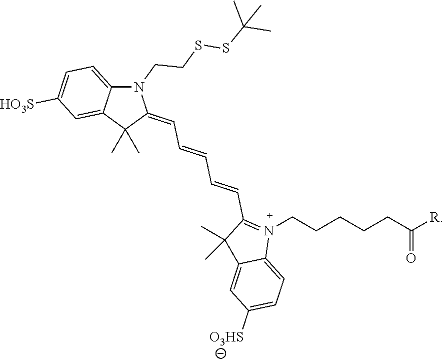

Upon reduction of the disulfide, the reducing agent-sensitive dye is converted to the structure:

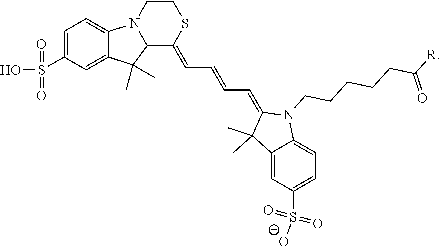

In some embodiments, the reducing agent-sensitive dye has the structure:

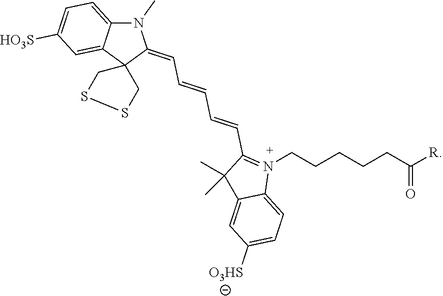

Upon reduction of the disulfide bond, the reducing agent-sensitive dye has the structure:

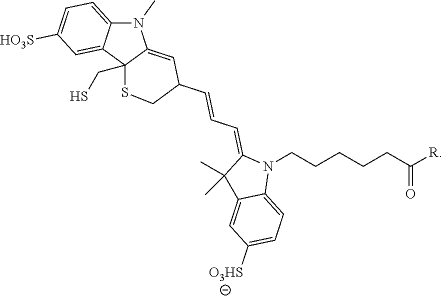

pH-Sensitive Fluorophores

In some embodiments, provided herein is a method comprising: contacting a biological sample with a composition comprising a first fluorescent nucleotide analogue or modified nucleotide and a second nucleotide analogue or modified nucleotide, wherein the second nucleotide analogue or modified nucleotide comprises a pH-sensitive dye in a non-fluorescent form; and performing a cycle of a nucleic acid sequencing reaction comprising a template nucleic acid in the biological sample, wherein the nucleic acid sequencing reaction comprises, sequentially: a) detecting a fluorescent intensity of the first fluorescent nucleotide analogue or modified nucleotide, wherein after detecting the first fluorescent nucleotide analogue or modified nucleotide, the second nucleotide analogue or modified nucleotide is modified to generate a fluorescent form of the second nucleotide analogue or modified nucleotide, and b) detecting a fluorescent intensity of the pH-sensitive dye of the second nucleotide analogue or modified nucleotide. In some embodiments, the second nucleotide analogue or modified nucleotide is modified to generate the fluorescent form of the second nucleotide analogue or modified nucleotide by protonation of the second nucleotide analogue or modified nucleotide.

In some embodiments, the pH-sensitive dye does not emit fluorescent in a basic environment, and emits fluorescence in an acidic environment. In some embodiments, the pH-sensitive dye does not emit fluorescent in an acidic environment, and emits fluorescence in a basic environment. In some embodiments, the pH of the imaging buffer decreases during one cycle of base-by-base sequencing, resulting in increased protonation of the pH-sensitive dye in the biological sample.

In some embodiments, the pH-sensitive dye has an associated pKa value, a pH value at which the fluorescence intensity is 50% of maximum. In some embodiments, the pH-sensitive dye has a pKA, and in step a) the imaging buffer has a pH that is above the pKA of the pH-sensitive dye. In some embodiments, in step b) the imaging buffer has a pH that is below the pKA of the pH-sensitive dye.

In some embodiments, the pH-sensitive fluorescent fluorophore or dye is a rhodamine derivative. In some embodiments, the pH-sensitive fluorescent dye has the following structure under acidic conditions:

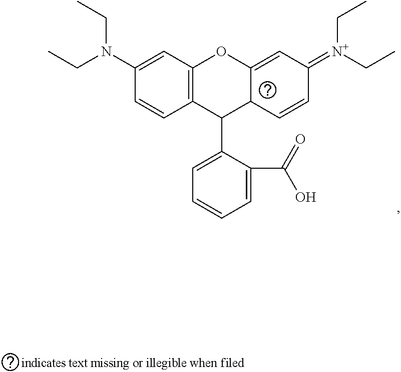 [SMILES: CCN(CC)c1ccc2c(c1)OC1=CC(=[N+](CC)CC)C=CC1C2c1ccccc1C(=O)O]

and the following structure under basic conditions:

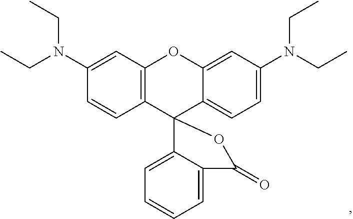 [SMILES: CCN(CC)c1ccc2c(c1)Oc1cc(N(CC)CC)ccc1C21OC(=O)c2ccccc21]

wherein the pH-sensitive dye is fluorescent in acidic conditions and non-fluorescent in basic conditions. In some embodiments, the pH-sensitive fluorescent dye under acidic conditions is a chloride salt, such as the chloride salt of rhodamine B. In some embodiments, the pH-sensitive fluorescent dye under acidic conditions is a sulfate salt, such as the sulfate salt of rhodamine B. In some embodiments, the pH-sensitive fluorescent dye under acidic conditions is a salt of an organic anion, such as the salt of rhodamine B with an organic anion, or a salt of an inorganic anion, such as the salt of rhodamine B with an inorganic anion.

Throughout the one or more cycles of nucleic acid sequencing reactions, an imaging buffer may be used in the samples or flow cells where the sequencing reaction is taking place. The imaging buffer has an associated pH. In some embodiments, the imaging buffer comprises an oxygen scavenger that releases a proton. In some embodiments, the pH of the imaging buffer decreases during the nucleic acid sequencing reaction. As the pH of the imaging buffer decreases, the reaction becomes more acidic, protonating the pH-sensitive fluorophore or pH-sensitive dyes. In some embodiments, the change in pH throughout the one or more cycles of nucleic acid sequencing reactions modifies the fluorophore.

In some embodiments, the pH-sensitive dye (e.g., pH-sensitive fluorophore) has a pKA, and the detecting of the second nucleotide analogue or modified nucleotide is performed in an imaging buffer having a pH above the pKA, and the detecting of the first fluorescent nucleotide analogue is performed in an imaging buffer having a pH that is below the pKA of the pH-sensitive dye.

In some embodiments, the pH-sensitive dye (e.g., pH-sensitive fluorophore) has a pKA, and during detecting of the first fluorescent nucleotide analogue or modified nucleotide the imaging buffer has a pH that is above the pKA of the pH-sensitive dye. In some embodiments, during detecting of the second fluorescent nucleotide, the imaging buffer has a pH that is below the pKA of the pH-sensitive dye.

### III. Nucleic Acid Sequencing with Selective Modification of Nucleotide Fluorescence

The sequencing methods described herein are useful for multi-color sequencing approaches where nucleotides with one or more modifiable fluorophore are used in the sequencing of a template nucleic acid. The template nucleic acid are “interrogated” by binding of a complementary nucleotide, or by incorporation of a complementary nucleotide into a priming strand bound to the template nucleic acid. For example, the sequencing methods described herein are applicable in situ sequencing applications (e.g., in situ sequencing of endogenous nucleic acid sequences and/or target-specific barcode sequences associated with target analytes of interest that are distributed within a cell or tissue sample). In some embodiments, the sequence of an endogenous nucleic acid or a target-specific barcode sequence is incorporated into a rolling circle amplification product in the cell or tissue sample, thereby providing multiple copies of the endogenous nucleic acid sequence or barcode sequence to be analyzed in the nucleic acid sequencing reaction. In some embodiments, the template nucleic acid is a rolling circle amplification product in the cell or tissue sample. Advantageously, the sequencing methods described herein allow for modification of the fluorescent fluorophores used to detect nucleotides while minimizing optical crowding compared to conventional methods for sequencing with nucleotides with static fluorescence.

In some aspects, the methods provided herein are useful for performing a cycle of a nucleic acid sequencing reaction on a template nucleic acid in the biological sample comprising nucleotides or nucleotide analogues with modifiable fluorescence.

In some embodiments, the sequencing methods described herein comprise contacting a template nucleic acid molecule with a first fluorescent nucleotide analogue or modified nucleotide and a second nucleotide analogue or modified nucleotide in a fluorescent form designed to bind to a portion of the template nucleic acid molecule. In some embodiments, after detecting the fluorescent intensity of the second dye, the second nucleotide analogue or modified nucleotide is modified to generate a non-fluorescent form of the second nucleotide analogue or modified nucleotide, and a fluorescent intensity of the first fluorescent nucleotide analogue or modified nucleotide is detected.

In some embodiments, the sequencing methods described herein comprise contacting a template nucleic acid molecule with a first fluorescent nucleotide analogue or modified nucleotide and a second nucleotide analogue or modified nucleotide in a non-fluorescent form designed to bind to a portion of the template nucleic acid molecule. In some embodiments, after detecting the fluorescent intensity of the first dye, the second nucleotide analogue or modified nucleotide is modified to generate a fluorescent form of the second nucleotide analogue or modified nucleotide, and a fluorescent intensity of the second fluorescent nucleotide analogue or modified nucleotide is detected.

In some embodiments, the fluorescent form of the nucleotide analogue or nucleotide comprises a quencher-attachment moiety. In some embodiments, a quencher is attached to the quencher-attachment moiety using click chemistry. In some embodiments, the quencher is attached using an azide-DBCO reaction, a tetrazine-TCO reaction, or an azide-alkyne reaction. In some embodiments, the fluorescent form of the nucleotide analogue or nucleotide transitions to a non-fluorescent form of the nucleotide analogue or nucleotide. In some embodiments, the non-fluorescent form of the nucleotide analogue or nucleotide comprises a quencher.

In some embodiments, the non-fluorescent form of the nucleotide analogue or nucleotide comprises a quencher. In some embodiments, a quencher is linked to the nucleotide via a cleavable linker. In some embodiments, the quencher is linked via a reduction-cleavable disulfide bond. In some embodiments, the linker is cleaved and/or the reaction is contacted with a reducing agent to cleave the disulfide bond. In some embodiments, the non-fluorescent form of the nucleotide analogue or nucleotide transitions to a fluorescent form of the nucleotide analogue or nucleotide. In some embodiments, the fluorescent form of the nucleotide analogue or nucleotide does not comprise a quencher and/or a disulfide bond.

In some embodiments, the fluorescent form of the nucleotide analogue or nucleotide comprises a reducing agent-sensitive dye comprising a disulfide moiety. In some embodiments, the reducing agent-sensitive fluorophore is a modified sulfo-Cy5 including a moiety that contains a disulfide bond. In some embodiments, the disulfide moiety is cleavable upon contact with a reducing agent, wherein the reducing agent-sensitive dye become non-fluorescent when the disulfide moiety is cleaved. In some embodiments, the nucleic acid sequencing reaction is contact with a reducing agent. In some embodiments, the fluorescent form of the nucleotide analogue or nucleotide transitions to a non-fluorescent form of the nucleotide analogue or nucleotide. In some embodiments, the non-fluorescent form of the nucleotide analogue or nucleotide does not comprises a disulfide moiety.

In some embodiments, the non-fluorescent form of the nucleotide analogue or nucleotide comprises a pH-sensitive fluorophore. In some embodiments, the pH-sensitive fluorophore is non-fluorescent in basic environments, and is fluorescent in acidic environments. In some embodiments, throughout the one or more cycles of nucleic acid sequencing reactions, the acidity of the reaction increases (e.g., pH decreases). In some embodiments, the pH-sensitive fluorophore is protonated. In some embodiments, the non-fluorescent form of the nucleotide analogue or nucleotide transitions to a fluorescent form of the nucleotide analogue or nucleotide.

In some embodiments, the fluorescent form of the nucleotide analogue or nucleotide comprises a pH-sensitive fluorophore. In some embodiments, the pH-sensitive fluorophore is fluorescent in basic environments, and is non-fluorescent in acidic environments. In some embodiments, throughout the one or more cycles of nucleic acid sequencing reactions, the acidity of the reaction increases (e.g., pH decreases). In some embodiments, the pH-sensitive fluorophore is deprotonated. In some embodiments, the fluorescent form of the nucleotide analogue or nucleotide transitions to a non-fluorescent form of the nucleotide analogue or nucleotide.

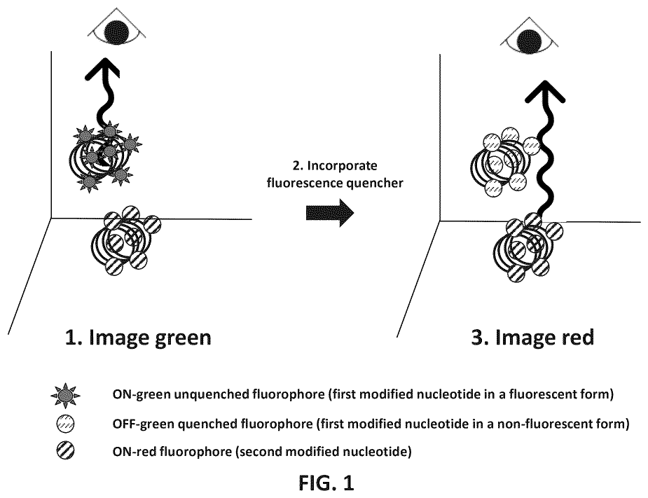

FIGS. 1-4 illustrate various examples of base-by-base sequencing according to the methods provided herein. In some embodiments, as shown in FIG. 1, the first fluorescent nucleotide analogue or modified nucleotide is modified from a fluorescent form (“ON”) (FIG. 1, left) to a non-quenched form (“OFF”) (FIG. 1, right) by selectively attaching a quencher to the first fluorescent nucleotide analogue or modified nucleotide. In some embodiments, the quencher is attached to the first fluorescent nucleotide analogue or modified nucleotide using click chemistry. In some embodiments, a cycle of base-by-base sequencing comprises detecting the first fluorescent nucleotide analogue or modified nucleotide before attaching the quencher. In some embodiments, after detecting, a fluorescence quencher is incorporated, and the fluorophore of the first fluorescent nucleotide analogue or modified nucleotide is quenched (FIG. 1, right). In some embodiments, the first fluorescent nucleotide analogue or modified nucleotide is contacted with the biological sample in a composition that also comprises a second nucleotide analogue or modified nucleotide, wherein the second nucleotide analogue or modified nucleotide remains “ON” throughout imaging and the sequencing process. In some embodiments, selectively quenching fluorescence of the first fluorescent nucleotide analogue or modified nucleotide allows improved optical resolution of signals corresponding to the first nucleotide analogue and the second nucleotide analogue. This is advantageous particularly in methods such as in situ sequencing in a tissue sample or matrix embedding a biological sample, wherein template nucleic acid molecules such as RCPs may produce optically overlapping signals. In some aspects, the present application addresses technical problems associated with optical crowding by allowing the signal of one fluorophore (associated with one of the bases in a base-by-base sequencing reaction) to be selectively masked (e.g., after detecting that fluorophore).

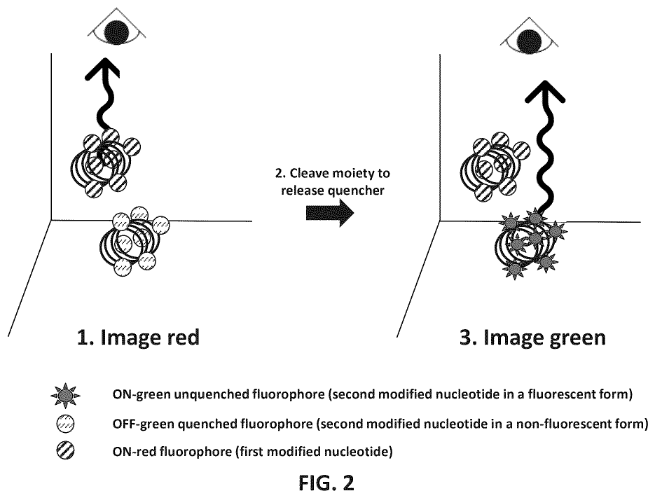

In some embodiments, as shown in FIG. 2, the second nucleotide analogue or modified nucleotide is modified from a non-fluorescent form (“OFF”) (FIG. 2, left) to a fluorescent form (“ON”) (FIG. 2, right) by selectively cleaving a quencher from the second nucleotide analogue or modified nucleotide. In some embodiments, the method comprises first detecting a first fluorescent nucleotide analogue or modified nucleotide wherein the second nucleotide analogue or modified nucleotide is in a non-fluorescent (e.g., quenched) form, and then cleaving the quencher from the second nucleotide analogue or modified nucleotide, and detecting the second nucleotide or nucleotide analogue. As in other embodiments disclosed herein, this is advantageous particularly in methods such as in situ sequencing in a tissue sample or matrix embedding a biological sample, wherein template nucleic acid molecules such as RCPs may produce optically overlapping signals. In some aspects, the present application addresses technical problems associated with optical crowding by allowing the signal of one fluorophore (associated with one of the bases in a base-by-base sequencing reaction) to be selectively unmasked (e.g., after detecting a signal from a different fluorophore).

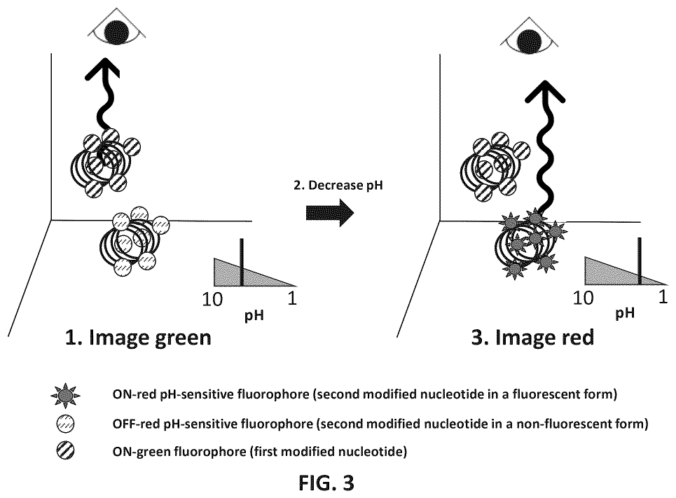

In some embodiments, as illustrated in FIG. 3, the second nucleotide or nucleotide analogue comprises a pH-sensitive dye. In some embodiments, the pH-sensitive dye becomes activated by protonation of the dye. In some embodiments, a greater percentage of the pH-sensitive dye molecules become activated in an acidic environment than in a basic environment (e.g., greater than 50%). In some embodiments, the method comprises decreasing the pH of the imaging buffer after detecting a first nucleotide analogue or modified nucleotide, and before detecting the second nucleotide analogue or modified nucleotide. In some embodiments, the imaging buffer used in a base-by-base sequencing reaction becomes more acidic as the biological sample is imaged. As shown in FIG. 3 (left panel), the pH-sensitive fluorophore is initially “OFF”. As the pH decreases, the pH-sensitive fluorophore fluoresces and is “ON” (FIG. 3, right). As in other embodiments disclosed herein, this is advantageous particularly in methods such as in situ sequencing in a tissue sample or matrix embedding a biological sample, wherein template nucleic acid molecules such as RCPs may produce optically overlapping signals. In some aspects, the present application addresses technical problems associated with optical crowding using a composition of nucleotides for base-by-base sequencing, wherein at least one of the nucleotides comprises a pH-sensitive dye, allowing for tunable fluorescence of the nucleotide comprising the pH-sensitive dye.

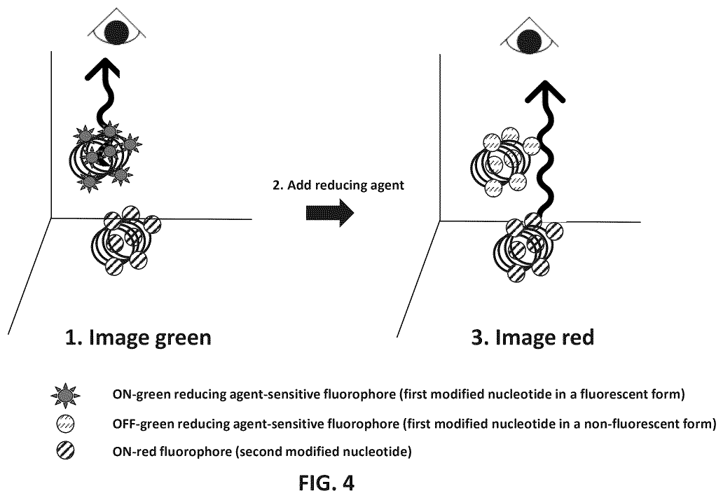

In some embodiments, as illustrated in FIG. 4, the first nucleotide or nucleotide analogue comprises a reducing agent-sensitive dye (e.g., reducing agent-sensitive fluorophore). In some embodiments, the reducing agent-sensitive dye transitions to a non-fluorescent form upon exposure to a reducing agent. In some embodiments, the method comprises adding the reducing agent after detecting the first nucleotide analogue or modified nucleotide, and before detecting the second nucleotide analogue or modified nucleotide. In some embodiments, the method comprises first detecting a first fluorescent nucleotide analogue or modified nucleotide wherein the first nucleotide analogue or modified nucleotide is in a fluorescent form, and then adding the reducing agent, and detecting the second nucleotide or nucleotide analogue. As shown in FIG. 4 (left panel), the reducing agent-sensitive fluorophore is initially “ON”. Upon exposure to the reducing agent, the reducing agent-sensitive fluorophore is reduced and is “OFF” (FIG. 4, right). As in other embodiments disclosed herein, this is advantageous particularly in methods such as in situ sequencing in a tissue sample or matrix embedding a biological sample, wherein template nucleic acid molecules such as RCPs may produce optically overlapping signals. In some aspects, the present application addresses technical problems associated with optical crowding by allowing the signal of one fluorophore (associated with one of the bases in a base-by-base sequencing reaction) to be selectively unmasked (e.g., after detecting a signal from a different fluorophore).

In some embodiments, the method comprises contacting the sample or the flow cell with a third nucleotide. In some embodiments, the method comprises contacting the sample or the flow cell with a fourth nucleotide. In some embodiments, the first, second, third, and fourth fluorescent forms of the nucleotide or nucleotide analogues emit fluorescence with distinguishable wavelengths. In some embodiments, the first, second, and third fluorescent forms of the nucleotide or nucleotide analogues emit fluorescence with distinguishable wavelengths, and the fourth nucleotide does not comprise a fluorophore.

In some embodiments, during the nucleic acid sequencing reaction, the nucleotides of a single identity (having a same nucleobase) are added or flowed in one at a time. For example, a first nucleotide is flowed in first, and a second nucleotide is flowed in second, sequentially and separately. In some embodiments, two nucleotides of two different identities are added or flowed in at the same time. For example, a first nucleotide and a second nucleotide are flowed in together. In some embodiments, three nucleotides of three different identities are added or flowed in at the same time. In some embodiments, four nucleotides of four different identities are added or flowed in at the same time.

### Template Nucleic Acid Molecules

In some embodiments, the template nucleic acid molecule includes a target analyte nucleic acid molecule (e.g., a DNA molecule, an RNA molecule, or an mRNA molecule). In some embodiments, the template nucleic acid includes a reporter oligonucleotide, such as a barcode.

In some embodiments, the template nucleic acid molecule is a DNA molecule. Examples of DNA template nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. In some embodiments, the DNA molecule is copied from another nucleic acid molecule (e.g., DNA or RNA such as mRNA).

In some embodiments, the template nucleic acid molecule is an RNA molecule. Examples of RNA template nucleic acid molecules include RNA molecules such as various types of coding and non-coding RNA. Examples of the different types of RNA molecules include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. In some embodiments, the RNA template nucleic acid molecule is copied from another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. In some embodiments, the RNA is small (e.g., less than 200 nucleic acid bases in length) or large (e.g., RNA greater than 200 nucleic acid bases in length). Examples of small RNAs include 5.85 ribosomal RNA (rRNA), 5S rRNA, tRNA, miRNA, siRNA, snoRNAs, piRNA, tRNA-derived small RNA (tsRNA), and small rDNA-derived RNA (srRNA). In some embodiments, the RNA is double-stranded RNA or single-stranded RNA. In some embodiments, the RNA is circular RNA. In some embodiments, the RNA is a bacterial rRNA (e.g., 16s rRNA or 23s rRNA).

In some embodiments, the template nucleic acid comprises a nucleic acid analyte derived from a biological sample and/or a reporter oligonucleotide or nucleic acid marker associated with an analyte from a biological sample. Such analytes can be or derived from any biological sample. In some embodiments, the template nucleic acid comprises a nucleic acid analyte and/or a reporter oligonucleotide or nucleic acid marker associated with an analyte present in a biological sample, and the template nucleic acid molecule is sequenced at a location in the biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, in some embodiments, a biological sample is obtained from a prokaryote such as a bacterium, an archaea, a virus, or a viroid. In some embodiments, a biological sample is obtained from non-mammalian organisms (e.g., a plant, an insect, an arachnid, a nematode, a fungus, or an amphibian). In some embodiments, a biological sample is obtained from a eukaryote, such as a tissue sample, a patient derived organoid (PDO) or patient derived xenograft (PDX). In some embodiments, a biological sample from an organism comprises one or more other organisms or components therefrom. For example, a mammalian tissue section may comprise a prion, a viroid, a virus, a bacterium, a fungus, or components from other organisms, in addition to mammalian cells and non-cellular tissue components. In some embodiments, subjects from which biological samples are obtained are healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, a template nucleic acid includes a reporter oligonucleotide or marker associated with the presence of an analyte (e.g., an endogenous analyte) in a sample. Such analytes may include nucleic acid analytes and/or non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Examples of analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In some embodiments, a template nucleic acid molecule may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

### Nucleotide Compositions for Sequencing

In some embodiments, methods and compositions disclosed herein are used to analyze any number of template nucleic acid molecules (e.g., nucleic acid analytes and/or analyte-associated barcode sequences) or fragments thereof. For example, in some embodiments, the number of analytes that are analyzed is at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of a sample (e.g., a cell sample or tissue sample) or tethered within individual features on a substrate (e.g., a flow cell surface).

In some embodiments, each cycle of a cyclic series of base-by-base sequencing reactions performed as part of the disclosed methods comprises contacting priming strands bound to template nucleic acid molecules with a composition comprising nucleotides. In some embodiments, each cycle of a cyclic series of base-by-base sequencing reactions comprises contacting the priming strand bound to a template nucleic acid molecule with a composition comprises a first nucleotide of a first nucleobase type and a second nucleotide of a second nucleobase type, wherein at least one of the first nucleotide and the second nucleotide is a modifiable nucleotide having modifiable fluorescence. In some embodiments, the modifiable nucleotide is a fluorescent nucleotide that is modifiable to generate a non-fluorescent form of the modifiable nucleotide. In some embodiments, the modifiable nucleotide is a non-fluorescent nucleotide that is modifiable to generate a fluorescent form of the modifiable nucleotide. In some embodiments, the first nucleotide and the second nucleotide are both modifiable nucleotides having modifiable fluorescence. In some embodiments, the composition further comprises a third nucleotide of a third nucleobase type and a fourth nucleotide of a fourth nucleobase type.

In some embodiments, nucleotides of a composition contacted with the primed template nucleic acid molecule(s) in each cycle of a multicycle sequencing process comprise A, T, U, C, and/or G. In some embodiments, nucleotides of a composition contacted with the primed template nucleic acid molecule(s) in each cycle of a multicycle sequencing process comprise A, T, C, and/or G.

In some embodiments, the composition comprising nucleotides contacted with the primed template nucleic acid molecule(s) is the same in each cycle of a multicycle sequencing process (e.g., the composition comprises the same sets of nucleotides in each cycle, wherein each set of nucleotides comprises the same selection of A, T, U, C, and/or G).

In some embodiments, the composition comprising nucleotides contacted with the primed template nucleic acid molecule(s) is different between at least 2 cycles of a multicycle sequencing process (e.g., the composition comprises different sets of nucleotides in different cycles, wherein the sets of nucleotides comprise a different selection of A, T, U, C, and/or G, and/or wherein the sets of nucleotides comprise a different selection fluorophores).

In some embodiments, a composition comprises 4 sets of nucleotides, wherein each set comprises a same nucleobase (e.g., selected from A, T, U, C, and/or G) that differs from the other sets, and wherein, for each set, nucleotides are labeled with a same fluorophore that differs from the other sets.

In some embodiments, the labeled and/or unlabeled nucleotides in the composition comprise modifiable labels (e.g., fluorophores) as described in detailed above (e.g., Section II). In some embodiments, the composition of nucleotides is a composition for sequencing-by-binding or sequencing-by-synthesis, and the method further comprises contacting the priming strands bound to template nucleic acids with a separate composition comprising nucleotides comprising modifiable fluorophores. In some embodiments, nucleotide composition with modifiable fluorophores contacted with the primed template nucleic acid molecule(s) in each cycle of a multicycle sequencing process comprise A, T, U, C, and/or G. In some embodiments, nucleotide composition with modifiable fluorophores contacted with the primed template nucleic acid molecule(s) in each cycle of a multicycle sequencing process comprise A, T, C, and/or G.

### Primers (or Primer Sequences)

In some instances, the disclosed methods for performing nucleic acid sequencing (e.g., in situ sequencing) can comprise the use of primer sequences that are complementary to, e.g., a subsequence (or primer binding site) that is part of an endogenous nucleic acid target sequence or a sequence (or primer binding site) that is located at or near a barcode (identifier) sequence associated with a target analyte. In some instances, a primer sequence may be designed to hybridize to a primer binding site associated with a single target analyte sequence and/or an associated target-specific barcode sequence. In some instances, a primer sequence may be designed to hybridize to a sequence (or primer binding site) that is associated with a plurality of target analyte sequences and/or associated target-specific barcode sequences (e.g., at least 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, or more than 1000 target analyte sequences and/or associated target-specific barcode sequences).

### Polymerases

Examples of polymerases that are used for performing the disclosed methods include, but are not limited to, DNA polymerases (e.g., Taq DNA polymerase), RNA polymerases, and/or reverse transcriptases.

In some embodiments, the polymerase is a DNA polymerase. Examples of DNA polymerases include Taq polymerase, 9° N-7 DNA polymerase (or variants thereof, for example, D141A/E143A/A485L), phi29 (φ29) polymerase, Klenow fragment, Bacillus stearothermophilus DNA polymerase (BST), T4 DNA polymerase, T7 DNA polymerase, and DNA polymerase I. In some embodiments, DNA polymerases that have been engineered or mutated to have desirable characteristics are employed. In some embodiments, the polymerase is phi29 DNA polymerase. In some embodiments, the polymerase is a DNA polymerase and the template nucleic acid molecule includes DNA. In some embodiments, the polymerase is a DNA polymerase and the nucleotide molecules include deoxyribonucleotides.

In some embodiments, the DNA polymerase is Taq polymerase or a functional variant thereof. Taq polymerase is a heat stable polymerase from Thermus aquaticus.

In some embodiments, the DNA polymerase is a 9° N-7 DNA polymerase or a functional variant thereof (e.g., D141A/E143A/A485L). 9° N-7 is a strain of Thermococcus sp.

In some embodiments, the DNA polymerase is DNA polymerase I or a functional fragment thereof (e.g., a Klenow fragment). Klenow fragment is an exonuclease deficient fragment of DNA polymerase I.

In some embodiments, the polymerase is a reverse transcriptase. Reverse transcriptases typically have RNA-dependent DNA polymerase activity and DNA-dependent DNA polymerase activity. Examples of reverse transcriptases include Moloney murine leukemia virus (MMLV) reverse transcriptase, HIV-1 reverse transcriptase, and avian myeloblastosis virus (AMV) reverse transcriptase. In some embodiments, the reverse transcriptase lacks (e.g., is mutated to lack) ribonuclease activity. In some embodiments, ribonuclease activity degrade template particularly during longer incubation times such as when reverse transcribing longer cDNAs. In some embodiments, the polymerase is a reverse transcriptase and the template nucleic acid molecule is an RNA molecule. In some embodiments, the polymerase is a reverse transcriptase and the nucleotide molecules include deoxyribonucleotide molecules.

In some embodiments, the reverse transcriptase is an MMLV reverse transcriptase or a functional variant thereof.

In some embodiments, the reverse transcriptase is an HIV-1 reverse transcriptase or a functional variant thereof.

In some embodiments, the polymerase is selected from Taq polymerase, a family B polymerase such as 9° N-7 DNA polymerase or a functional variant thereof (e.g., D141A/E143A/A485L), and a Klenow fragment of DNA polymerase I.

### Base-Calling

As noted elsewhere herein, the disclosed methods for performing nucleic acid sequencing (e.g., in situ sequencing) may comprise inferring the sequence of a template nucleic acid molecule from a series of optical signals (e.g., fluorescence signals) detected in images acquired during a repetitive series of sequencing reaction cycles in a process referred to as “base-calling”. The interplay of sequencing chemistry, opto-fluidics hardware, optical sensors, and signal processing software utilized in sequencing platforms affects the types of errors made during sequencing (see, e.g., Lederberger et al. (2011), “Base-calling for next-generation sequencing platforms”, Brief Bioinform. 12(5): 489-497). The characterization of errors associated with the sequencing process and implementation of chemistry-, imaging-, and/or signal processing software-based methods for minimizing sequence errors are thus important for maximizing the accuracy of sequencing results.

In four-color sequencing-by-synthesis methods, for example, a set of four images—one image for each of four detection channels corresponding to the emission wavelengths for four fluorophores used to label the reversibly terminated nucleotides—are acquired in each sequencing cycle. Processing of the images to detect fluorescence intensity signals produces an intensity quadruple for the location of each sequencing colony on a flow cell surface (or the location of each target analyte, or amplified representation thereof (e.g., an RCP) in the case of in situ sequencing), where each value represents the intensity of the fluorescence signal for the detection channels corresponding to A, C, G and T. Ideally, the channel in which the maximum intensity occurs would be the base that is “called” for a given RCP or sequencing colony (or target analyte) in a given cycle. However, the chemical processes involved in sequencing are imperfect, leading to errors in base-calling (see, e.g., Cacho, et al. (2016), “A Comparison of Base-calling Algorithms for Illumina Sequencing Technology”, Briefings in Bioinformatics 17(5):786-795). In some sequencing-by-synthesis (SBS) platforms, for example, sources of error may include phasing (or lagging; e.g., where the primed template nucleic acid molecules at one or more locations fail to incorporate the next base due to variation in polymerase reaction kinetics), pre-phasing (or leading; e.g., where more than one nucleotide is incorporated in a single cycle due to, e.g., impurities in the reversibly terminated nucleotides), signal decay (due to, e.g., photobleaching and/or loss of template nucleic acid during the sequencing process), and cross-talk (e.g., when two or more fluorophore emission spectra overlap, which may cause a positive correlation between signal intensities measured in the corresponding detection channels).

A variety of statistical approaches have been developed to correct for, or minimize, such errors and generate more accurate base-calls. Examples include, but are not limited to, AYB (Goldman Group, European Molecular Biology Laboratory—European Bioinformatics Institute, Cambridgeshire, UK), and Bustard (Illumina, Inc., San Diego, CA).

### Sequence Reads

The output of the base-calling process applied to optical signals detected in a series of images of a biological sample or flow cell surface acquired during a cycling sequencing process consists of a plurality of sequence reads, e.g., the nucleotide sequences determined for all or a portion of a template nucleic acid molecule (e.g., an endogenous nucleic acid analyte or a barcode sequence associated with a target analyte).

In some instances, the sequence reads generated using the disclosed methods for nucleic acid sequencing may comprise sequence reads of at least about 10, 20, 30, 40, 50, 60, 70, 80, 90, 100 or more nucleotides or base pairs of the template nucleic acid sequences. In some instances, the sequence reads generated using the disclosed methods may comprise sequence reads of at least about 100, 120, 140, 160, 180, 200, 220, 240, 260, 280, 300, 320, 340, 360, 380, 400, or more than 400 nucleotides or base pairs of the template nucleic acid sequences.

In some instances, the disclosed methods for in situ or flow cell sequencing may generate at least about 100, 200, 300, 400, 500, 600, 700, 800, 900, 1,000, or more sequencing reads per run. In some instances, the disclosed method may generate at least about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 10⁶, 5×10⁶, 10⁷, or more than 10⁷ sequencing reads per run.

### Sequence Assembly & Alignment

In some instances, the disclosed methods for nucleic acid sequencing comprise assembly of longer template nucleic acid sequences, e.g., genome fragments or whole genomes, from a plurality of relatively short sequence reads. Sequence assembly may be performed by identifying the overlapping sequences from multiple short sequence reads to assemble longer, contiguous sections of sequence.

In some instances, the disclosed methods for nucleic acid sequencing comprise identifying a code word corresponding to a sequence read or an assembled sequence, where the code word is one of a plurality of code words in a codebook that includes assignment of each of the plurality of code words to a target analyte of interest. The sequence read or assembled sequence may thus be used to identify a specific target analyte (based on the corresponding code word) in, e.g., a multiplexed in situ detection or sequencing assay.

In some instances, the disclosed methods for nucleic acid sequencing comprise alignment of sequence reads and/or assembled sequences to a known reference sequence or consensus sequence (e.g., the GRCh38 human reference genome (Genome Reference Consortium)) from the same or a similar organism. Alignment to a reference sequence or consensus sequence may be used to identify gaps, errors, or variants in the assembled sequence. Any of a variety of bioinformatics software programs may be used to assemble longer sequences from relatively short sequence reads. Examples include, but are not limited to, DBG2OLC (see, e.g., Ye et al. (2016), “DBG2OLC: Efficient Assembly of Large Genomes Using Long Erroneous Reads of the Third Generation Sequencing Technologies”, Scientific Reports 6:31900), SPAdes (see, e.g., Bankevich et al. (2012), “SPAdes: A New Genome Assembly Algorithm and Its Applications to Single-Cell Sequencing”, J. Computational Biol. 19(5):455-477), SparseAssembler (see, e.g., Ye et al. (2012), “Exploiting Sparseness in de novo Genome Assembly”, BMC Bioinformatics 13(Suppl 6):S1), Fermi (see, e.g., Li (2012), “Exploring Single-Sample SNP and INDEL Calling with Whole-Genome de novo Assembly”, Bioinformatics 28(14):1838-1844), and String Graph Assembler (SGA) (see, e.g., Simpson et al. (2012), “Efficient de novo Assembly of Large Genomes Using Compressed Data Structures”, Genome Res. 22: 549-556).

### Workflows for Performing Sequencing

In some embodiments, the method comprises performing one or more cycles of a base-by-base sequencing reaction using a modifiable nucleotide molecule with modifiable fluorescence. In some embodiments, the method includes first detecting the modifiable nucleotide while in a fluorescent form and then modifying the modifiable nucleotide to generate a non-fluorescent form prior to detecting an additional nucleotide. In some embodiments, the method includes first detecting a first nucleotide while a modifiable nucleotide is in a non-fluorescent form, and then modifying the modifiable nucleotide to generate a fluorescent form prior to detecting the modifiable nucleotide.

In some embodiments, a given cycle of the base-by-base sequencing reaction comprises contacting the biological sample with a composition comprising a first fluorescent nucleotide analogue or modified nucleotide and a second nucleotide analogue or modified nucleotide in a fluorescent form and, sequentially: a) detecting a fluorescent intensity of the second nucleotide analogue or modified nucleotide, wherein after detecting the fluorescent intensity of the second dye, the second nucleotide analogue or modified nucleotide is modified to generate a non-fluorescent form of the second nucleotide analogue or modified nucleotide, and b) detecting a fluorescent intensity of the first fluorescent nucleotide analogue or modified nucleotide.

In some embodiments, a given cycle of the base-by-base sequencing reaction comprises, sequentially: a) contacting the biological sample with a composition comprising a first fluorescent nucleotide and a second nucleotide fluorescent nucleotide, wherein the second fluorescent nucleotide is convertible to a non-fluorescent form; b) detecting a fluorescent intensity of the second fluorescent nucleotide; c) converting the second fluorescent nucleotide to the non-fluorescent form of the second nucleotide; and d) detecting a fluorescent intensity of the first nucleotide.

In some embodiments, the second fluorescent nucleotide comprises an attachment moiety for attaching a quencher, and converting the second fluorescent nucleotide to the non-fluorescent form comprises attaching the quencher. In some embodiment, the quencher is a broad spectrum quencher. In some embodiments, the attachment moiety is a click chemistry attachment moiety.

In some embodiments, the second fluorescent nucleotide comprises a pH-sensitive fluorophore, and converting the second fluorescent nucleotide to the non-fluorescent form comprises modifying the pH to deactivate the fluorescence of the pH-sensitive fluorophore. In some embodiments, modifying the pH comprises decreasing the pH. In some embodiments, modifying the pH comprises increasing the pH.

In some embodiments, the second fluorescent nucleotide comprises a reducing agent-sensitive fluorophore, and converting the second fluorescent nucleotide to the non-fluorescent form comprises modifying the reducing conditions to deactivate the fluorescence of the reducing agent-sensitive fluorophore. In some embodiments, modifying the reducing conditions comprises removing a reducing agent from the buffer. In some embodiments, modifying the reducing conditions comprises adding a reducing agent to the buffer.

In some embodiments, a given cycle of the base-by-base sequencing reaction comprises contacting the biological sample with a composition comprising a first fluorescent nucleotide analogue or modified nucleotide and a second nucleotide analogue or modified nucleotide, wherein the second nucleotide analogue or modified nucleotide is in a non-fluorescent form and, sequentially: a) detecting a fluorescent intensity of the first fluorescent nucleotide analogue or modified nucleotide, wherein after detecting the first fluorescent nucleotide analogue or modified nucleotide, the second nucleotide analogue or modified nucleotide is modified to generate a fluorescent form of the second nucleotide analogue or modified nucleotide, and b) detecting a fluorescent intensity of the second nucleotide analogue or modified nucleotide.

In some embodiments, a given cycle of the base-by-base sequencing reaction comprises, sequentially: a) contacting the biological sample with a composition comprising (i) a first nucleotide comprising a fluorescent nucleotide and (ii) a second nucleotide, wherein the second nucleotide is non-fluorescent and convertible to a fluorescent form; b) detecting a fluorescent intensity of the first nucleotide; c) converting the second nucleotide to the fluorescent form; and d) detecting a fluorescent intensity of the second nucleotide.

In some embodiments, the second nucleotide is attached to a quencher, and converting the second nucleotide to the fluorescent form comprises removing the quencher. In some embodiment, the quencher is a broad spectrum quencher. In some embodiments, the second nucleotide is attached to the quencher via a reducing agent-cleavable linker, and the removing comprises exposing the second nucleotide to a reducing agent. In some embodiments, the reducing agent-cleavable linker is selected from a disulfide linker and an O-azidomethyl linker.

In some embodiments, the second nucleotide comprises a pH-sensitive fluorophore, and converting the second nucleotide to the fluorescent form comprises modifying the pH to activate the fluorescence of the pH-sensitive fluorophore. In some embodiments, modifying the pH comprises decreasing the pH. In some embodiments, modifying the pH comprises increasing the pH.

In some embodiments, the second nucleotide comprises a reducing agent-sensitive fluorophore, and converting the second nucleotide to the fluorescent form comprises modifying the reducing conditions to activate the fluorescence of the reducing agent-sensitive fluorophore. In some embodiments, modifying the reducing conditions comprises removing a reducing agent from the buffer. In some embodiments, modifying the reducing conditions comprises adding a reducing agent to the buffer.

In some embodiments, the disclosed methods further comprise processing optical signals (e.g., fluorescence signals) detected in images (e.g., fluorescence images) acquired during the cyclic series of base-by-base sequencing reactions to detect the presence or absence of complementary nucleotides in each sequencing cycle at the locations of each of a plurality of template nucleic acid molecules (i.e., the locations corresponding to each of a plurality of target analyte molecules and/or their associated target-specific barcode sequences), thereby enabling inference of the nucleotide sequence of the plurality of template nucleic acid molecules (e.g., the plurality of target analyte molecules and/or associated target-specific barcode sequences).

In some embodiments, the cyclic series of base-by-base sequencing reactions comprises performing at least 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, or more than 50 cycles of the base-by-base sequencing reaction.

In some embodiments, each cycle of base-by-base sequencing further comprises a first wash step following the contacting step to remove unbound polymerase and nucleotides. In some embodiments, the first wash step comprises, for example, use of the same buffer used for contacting the primed template nucleic acid with a polymerase and a composition comprising nucleotides (but without the polymerase and composition). In some embodiments, the first wash buffer may not include KCl and/or may include little to no DMSO. In some embodiments, the first wash buffer is similar to those used for wash buffers as used in wash steps of a Western blot (e.g., a wash buffer added in a Western blot after binding a primary antibody but washing prior to incubation with a secondary antibody, such as PBST). PBST is a phosphate-buffered saline with a low-concentration of detergent, such as 0.05% to 0.1% Tween.

In some embodiments, each cycle of base-by-base sequencing further comprises removing the fluorophore from incorporated nucleotides following the detection step. In some embodiments, the fluorophore is attached to the incorporated nucleotide via a cleavable linker. In some embodiments, each cycle of base-by-base sequencing further comprises cleaving the linker between a fluorophore and the incorporated nucleotide, if the incorporated nucleotide comprises a fluorophore. Exemplary cleavable linkers and reaction conditions for cleaving such linkers are described elsewhere herein.

In some embodiments, each cycle of base-by-base sequencing further comprises photobleaching the fluorophore of the incorporated nucleotide following the detection step. In some embodiments, the fluorophore of the nucleotide is unable to fluoresce after photobleaching. Any suitable photobleaching methods can be implemented. In some embodiments, the sample is exposed to a light source until the signal emitted by the fluorophore is eliminated.

In some embodiments, the detection step comprises the use of an optical imaging technique (e.g., a fluorescence imaging technique) and real time or post-processing measurement of optical signals (e.g., fluorescence signals or the absence thereof) associated with the presence of a specific nucleotide at a plurality of locations corresponding to a plurality of target analytes distributed throughout the biological sample or tethered to specific locations on a substrate surface (e.g., a flow cell surface).

In some embodiments, the template nucleic acid molecule comprises an endogenous nucleic acid molecule (e.g., a DNA molecule, an RNA molecule, or an mRNA molecule) that has been reverse transcribed, amplified, and/or extracted from a biological sample (e.g., a cell sample or tissue sample).

In some embodiments, the template nucleic acid molecule comprises a barcode sequence (e.g., a nucleic acid barcode sequence) associated with a target analyte of interest (e.g., using the barcoding methods described elsewhere herein) that has been reverse transcribed, amplified, and/or extracted from a biological sample (e.g., a cell sample or tissue sample).

In some embodiments, the method further comprises hybridizing a circularizable probe to a target analyte (or to a labeling agent bound to the target analyte), ligating the circularizable probe to form a circularized probe, and performing rolling circle amplification of the circularized probe to generate the template nucleic acid molecule. In some embodiments, for example, the circularizable probe is a padlock probe sequence.

The disclosed sequencing methods may be applied to both in situ sequencing and flow cell sequencing applications, where the sequencing reactions described involving use of nucleotides with modifiable fluorescence are used rather than a conventional sequencing-by-synthesis (SBS), or sequencing-by-binding methods.

In some embodiments, the template nucleic acid molecule is sequenced in situ in a cell sample or tissue sample. In some embodiments, the cell sample comprises a layer of cells deposited on a surface.

In the case of in situ sequencing, in some embodiments, the disclosed methods comprise performing all or a subset of the following steps (in addition to a cyclic series of base-by-base sequencing reactions involving use of nucleotides with modifiable fluorescence):
(i) preparing the biological sample (e.g., by fixing, sectioning, embedding, and/or clearing a cell or tissue sample, as described elsewhere herein);
(ii) contacting target analytes (e.g., target nucleic acid analytes and/or protein analytes) within the prepared sample with target-specific probes, as described elsewhere herein. In some embodiments, the target-specific probes comprise, e.g., target-specific linear and/or circularizable nucleic acid probes (e.g., padlock probes) designed to hybridize directly or indirectly to specific target nucleic acid analytes. In some embodiments, the target-specific linear and/or circularizable nucleic acid probes comprise primer binding sites and/or target-specific barcode (or identifier) sequences. In some embodiments, the target-specific probes comprise, e.g., target-specific antibodies designed to bind to specific target protein analytes, where the antibodies are conjugated to nucleic acid sequences. In some embodiments, the conjugated nucleic acid sequences comprise primer binding sites and/or target-specific barcode (or identifier) sequences.

In some embodiments, the target nucleic acid analytes comprise RNA molecules and the in situ sequencing method further includes performing a reverse transcription reaction to create cDNA copies of RNA target molecules.

In some embodiments, the in situ sequencing method further includes (iv) amplifying the probed target analyte molecules and/or their associated target-specific barcode sequences (e.g., using rolling circle amplification (RCA) in the case that target-specific circularizable probes were used to probe target analyte molecules and/or associated barcode sequences). In some embodiments, the in situ sequencing method further includes (v) contacting the amplified target nucleic acid analytes and/or associated target-specific barcode sequences with sequencing primers designed to hybridize directly or indirectly to the target nucleic acid analytes and/or their associated target-specific barcode sequences.

### IV. Biological Samples & Sample Preparation

A sample disclosed herein can be or derived from any biological sample. The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some instances, the biological sample may be provided on a substrate. In some instances, a substrate herein can be any support that is insoluble in aqueous liquid and which allows for positioning of biological samples, analytes, features, and/or reagents (e.g., probes) on the support. In some instances, a biological sample can be attached to a substrate. Attachment of the biological sample can be irreversible or reversible, depending upon the nature of the sample and subsequent steps in the analytical method. In certain instances, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some instances, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some instances, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some instances, the biological sample is prepared using formalin-fixation and paraffin-embedding (FFPE), which are established methods. In some instances, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some instances, the methods provided herein comprise one or more post-fixing (also referred to as post-fixation) steps. In some instances, one or more post-fixing step is performed after contacting a sample with a polynucleotide disclosed herein, e.g., one or more probes such as a circular or padlock probe. In some instances, one or more post-fixing step is performed after a hybridization complex comprising a probe and a target is formed in a sample. In some instances, one or more post-fixing step is performed prior to a ligation reaction disclosed herein.

In some instances, a method disclosed herein comprises de-crosslinking the reversibly cross-linked biological sample. The de-crosslinking does not need to be complete. In some instances, only a portion of crosslinked molecules in the reversibly cross-linked biological sample are de-crosslinked and allowed to migrate.

In some instances, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some instances, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some instances, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some instances, DNase and RNase inactivating agents or inhibitors such as proteinase K, and/or chelating agents such as EDTA, can be added to the sample. For example, a method disclosed herein comprises a step for increasing accessibility of a nucleic acid for binding, e.g., a denaturation step to open up DNA in a cell for hybridization by a probe. For example, proteinase K treatment may be used to free up DNA with proteins bound thereto.

### Embedding

In some instances, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample. Biological samples can include analytes (e.g., protein, RNA, and/or DNA) embedded in a 3D matrix. In some instances, amplicons (e.g., rolling circle amplification products) derived from or associated with analytes (e.g., protein, RNA, and/or DNA) can be embedded in a 3D matrix. In some instances, a 3D matrix comprises a network of natural molecules and/or synthetic molecules that are chemically and/or enzymatically linked, e.g., by crosslinking. In some instances, a 3D matrix comprises a synthetic polymer. In some instances, a 3D matrix comprises a hydrogel.

In some embodiments, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some instances, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample.

In some instances, the biological sample is immobilized in the hydrogel via cross-linking of the polymer material that forms the hydrogel. Cross-linking can be performed chemically and/or photochemically, or alternatively by any other suitable hydrogel-formation method.

In some instances, the biological sample is reversibly cross-linked prior to or during an in situ assay. In some embodiments, the analytes, polynucleotides and/or amplification product (e.g., amplicon) of an analyte or a probe bound thereto can be anchored to a polymer matrix. For example, the polymer matrix can be a hydrogel. In some instances, one or more of the polynucleotide probe(s) and/or amplification product (e.g., amplicon) thereof is/are modified to contain functional groups that can be used as an anchoring site to attach the polynucleotide probes and/or amplification product to a polymer matrix. In some instances, a modified probe comprising oligo dT may be used to bind to mRNA molecules of interest, followed by reversible or irreversible crosslinking of the mRNA molecules.

In some instances, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some instances, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some instances, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

In some instances, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some instances, hydrogel formation occurs after contacting one or more second materials during formation of the substrate. Hydrogel formation can also occur within a structure (e.g., wells, ridges, projections, and/or markings) located on a substrate.

In some instances, hydrogel formation on a substrate occurs before, contemporaneously with, or after probes are provided to the sample. For example, hydrogel formation can be performed on the substrate already containing the probes.

In some instances, hydrogel formation occurs within a biological sample. In some embodiments, a biological sample (e.g., tissue section) is embedded in a hydrogel. In some instances, hydrogel subunits are infused into the biological sample, and polymerization of the hydrogel is initiated by an external or internal stimulus.

In instances in which a hydrogel is formed within a biological sample, functionalization chemistry can be used. In some instances, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some instances, hydrogel formation within a biological sample is permanent. For example, biological macromolecules can permanently adhere to the hydrogel allowing multiple rounds of interrogation. In some instances, hydrogel formation within a biological sample is reversible. In some instances, HTC reagents are added to the hydrogel before, contemporaneously with, and/or after polymerization. In some instances, a cell labeling agent is added to the hydrogel before, contemporaneously with, and/or after polymerization. In some instances, a cell-penetrating agent is added to the hydrogel before, contemporaneously with, and/or after polymerization.

In some instances, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some instances, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some instances, a hydrogel-embedded sample is stored before or after clearing of hydrogel, in a medium (e.g., a mounting medium, methylcellulose, or other semi-solid mediums).

In some instances, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some instances, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some instances, the sample is isometrically expanded to at least 2× and less than 20× of its non-expanded size.

### Staining and Immunohistochemistry (IHC)

To facilitate visualization, biological samples can be stained using a wide variety of stains and staining techniques. In some instances, for example, a sample can be stained using any number of stains and/or immunohistochemical reagents. One or more staining steps may be performed to prepare or process a biological sample for an assay described herein or may be performed during and/or after an assay. In some instances, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some instances, cells in the sample can be segmented using one or more images taken of the stained sample.

In some instances, the stain is performed using a lipophilic dye. In some examples, the staining is performed with a lipophilic carbocyanine or aminostyryl dye, or analogs thereof (e.g., DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some instances, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some instances, the sample can be stained using Romanowsky stain, including Wright's stain, Jenner's stain, Can-Grunwald stain, Leishman stain, and Giemsa stain.

In some instances, biological samples can be destained. Any suitable methods of destaining or discoloring a biological sample may be utilized and generally depend on the nature of the stain(s) applied to the sample. For example, in some instances, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### V. Analytes

A biological sample may comprise one or a plurality of analytes of interest. Methods for performing multiplexed assays to analyze two or more different analytes in a single biological sample are provided.

The methods and compositions disclosed herein can be used to detect and analyze a wide variety of different analytes. In some embodiments, an analyte can include any biological substance, structure, moiety, or component to be analyzed. In some embodiments, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

### Labelling Agents

In some instances, provided herein are methods and compositions for analyzing endogenous analytes (e.g., RNA, ssDNA, cell surface or intracellular proteins, and/or metabolites) in a sample using one or more labeling agents. In some instances, an analyte labeling agent may include an agent that interacts with an analyte (e.g., an endogenous analyte in a sample). In some instances, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some instances, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some instances, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some instances, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some instances, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of non-limiting examples of labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some instances, an analyte binding moiety includes one or more antibodies or epitope-binding fragments thereof. The antibodies or epitope-binding fragments including the analyte binding moiety can specifically bind to a target analyte. In some instances, the analyte is a protein (e.g., a protein on a surface of the biological sample (e.g., a cell) or an intracellular protein). In some instances, a plurality of analyte labeling agents comprising a plurality of analyte binding moieties bind a plurality of analytes present in a biological sample. In some instances, the plurality of analytes includes a single species of analyte (e.g., a single species of polypeptide). In some instances in which the plurality of analytes includes a single species of analyte, the analyte binding moieties of the plurality of analyte labeling agents are the same. In some instances in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some instances, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some embodiments, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some instances, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some instances, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### Detectable Labels

In some instances, one or more hybridization probes or one or more nucleotides (or analogs thereof) can be labeled with distinguishing and/or detectable tags or labels. The tags may be distinguishable by means of their differences in fluorescence, Raman spectrum, charge, mass, refractive index, luminescence, length, or any other measurable property. The tag may be attached to one or more different positions on the nucleotide, so long as the fidelity of binding to the polymerase-nucleic acid complex is sufficiently maintained to enable identification of the complementary base on the template nucleic acid correctly. In some instances, the tag is attached to the nucleobase of the nucleotide. Alternatively, a tag is attached to the gamma phosphate position of the nucleotide.

Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some instances, the detectable label is bound to another moiety, for example, a nucleotide or nucleotide analog, and can include a fluorescent, a colorimetric, or a chemiluminescent label.

In some instances, a detectable label can be attached to another moiety, for example, a nucleotide or nucleotide analog. In some instances, one or more nucleotides can be labeled with a cleavable detectable tag or label. For example, the non-terminating fluorescently labeled nucleotides can include a DBCO-nucleotide conjugated to fluorescent compound with a disulfide linker. In some instances, a non-terminating fluorescently labeled nucleotide is incorporated into the strand without termination, and after imaging, the linker can be cleaved to remove fluorescent label. In some instances, a DBCO-nucleotide (e.g., 5-DBCO-PEG₄-UTP) can undergo a click reaction with the cleavable linker conjugated to a fluorescent label (e.g., cleavable linker-ATTO647N), and a disulfide group can be cleaved by tris(2-carboxyethyl)phosphine (TCEP) reduction together with 3′-O-azidomethyl-dNTP.

The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

### Generation of Products

In some embodiments, sequencing methods provided herein include sequencing a template nucleic acid molecule. In some embodiments, the template nucleic acid molecule is a product (e.g., a hybridization product, a ligation product, an extension product (e.g., by a DNA or RNA polymerase), a replication product, a transcription/reverse transcription product, and/or an amplification product such as a rolling circle amplification (RCA) product) of an endogenous analyte of a biological sample. In some instances, provided herein are methods and compositions for analyzing one or more products of an endogenous analyte and/or a labeling agent in a biological sample. In some instances, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some instances, a labeling agent that directly or indirectly binds to an analyte in the biological sample is analyzed. In some instances, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### (a) Hybridization

In some instances, a hybridization product comprising the pairing of substantially complementary or complementary nucleic acid sequences within two different molecules can be analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Non-limiting examples of barcoded probes or probe sets may be based on a padlock probe (circularizable probe), a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

### (b) Ligation

In some instances, a ligation product of an endogenous analyte and/or a labeling agent can be analyzed. In some instances, the ligation product is formed between two or more endogenous analytes. In some instances, the ligation product is formed between two or more labeling agents. In some instances, the ligation product is an intramolecular ligation of an endogenous analyte. In some instances, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some instances, provided herein is a probe or probe set capable of DNA-templated ligation, such as from a cDNA molecule. See, e.g., U.S. Pat. No. 8,551,710, which is hereby incorporated by reference in its entirety. In some instances, provided herein is a probe or probe set capable of RNA-templated ligation. See, e.g., U.S. Pat. Pub. 2020/0224244 which is hereby incorporated by reference in its entirety. In some instances, the probe set is a SNAIL probe set. See, e.g., U.S. Pat. Pub. 20190055594, which is hereby incorporated by reference in its entirety. In some instances, provided herein is a multiplexed proximity ligation assay. See, e.g., U.S. Pat. Pub. 20140194311 which is hereby incorporated by reference in its entirety. In some instances, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some instances, a circular probe can be indirectly hybridized to the target nucleic acid. In some instances, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some instances, the ligation involves chemical ligation (e.g., click chemistry ligation). In some instances, the chemical ligation involves template dependent ligation. In some instances, the chemical ligation involves template independent ligation. In some instances, the click reaction is a template-independent reaction (see, e.g., Xiong and Seela (2011), J. Org. Chem. 76(14): 5584-5597, incorporated by reference herein in its entirety). In some instances, the click reaction is a template-dependent reaction or template-directed reaction. In some instances, the template-dependent reaction is sensitive to base pair mismatches such that reaction rate is significantly higher for matched versus unmatched templates. In some instances, the click reaction is a nucleophilic addition template-dependent reaction. In some instances, the click reaction is a cyclopropane-tetrazine template-dependent reaction.

In some instances, the ligation involves enzymatic ligation. In some instances, the enzymatic ligation involves use of a ligase. In some embodiments, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some instances, the ligase is a T4 RNA ligase. In some instances, the ligase is a splintR ligase. In some instances, the ligase is a single stranded DNA ligase. In some instances, the ligase is a T4 DNA ligase. In some instances, the ligase is a ligase that has an DNA-splinted DNA ligase activity. In some instances, the ligase is a ligase that has an RNA-splinted DNA ligase activity.

In some instances, the ligation herein is a direct ligation. In some instances, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some instances, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe (e.g., padlock probe), or target nucleic acid. The gap may be a gap of 1 to 60 nucleotides or a gap of 1 to 40 nucleotides or a gap of 3 to 40 nucleotides. In specific instances, the gap may be a gap of about 1, 2, 3, 4, 5, 6, 7, 8, 9 or 10 or more nucleotides, of any integer (or range of integers) of nucleotides in between the indicated values. In some instances, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some instances, the ligation herein is preceded by gap filling. In other instances, the ligation herein does not require gap filling.

In some instances, ligation of the polynucleotides produces polynucleotides with melting temperature higher than that of unligated polynucleotides. Thus, In some embodiments, ligation stabilizes the hybridization complex containing the ligated polynucleotides prior to subsequent steps, comprising amplification and detection.

In some embodiments, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some instances, the ligation herein is a proximity ligation of ligating two (or more) nucleic acid sequences that are in proximity with each other, e.g., through enzymatic means (e.g., a ligase). In some instances, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some instances, a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) can be analyzed.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally includes any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some instances, a product of an endogenous analyte and/or a labeling agent is an amplification product of one or more polynucleotides, for instance, a circular probe or circularizable probe or probe set. In some instances, the amplifying is achieved by performing rolling circle amplification (RCA). In other instances, a primer that hybridizes to the circular probe or circularized probe is added and used as such for amplification. In some instances, the RCA comprises a linear RCA, a branched RCA, a dendritic RCA, or any combination thereof.

In some instances, the amplification is performed at a temperature between or between about 20° C. and about 60° C. In some instances, the amplification is performed at a temperature between or between about 30° C. and about 40° C. In some embodiments, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some instances, upon addition of a DNA polymerase in the presence of appropriate dNTP precursors and other cofactors, a primer is elongated to produce multiple copies of the circular template. This amplification step can utilize isothermal amplification or non-isothermal amplification. In some instances, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). RCA may be performed using a strand-displacing polymerase. Non-limiting examples of polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some embodiments, DNA polymerases that have been engineered or mutated to have desirable characteristics can be employed. In some instances, the polymerase is phi29 DNA polymerase.

In some embodiments, during the amplification step, modified nucleotides can be added to the reaction to incorporate the modified nucleotides in the amplification product (e.g., nanoball). Non-limiting examples of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some embodiments, the amplification products comprises a modified nucleotide, such as an amine-modified nucleotide. In some instances, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some embodiments, the polynucleotides and/or amplification product (e.g., amplicon) can be anchored to a polymer matrix. For example, the polymer matrix can be a hydrogel. In some instances, one or more of the polynucleotide probe(s) can be modified to contain functional groups that can be used as an anchoring site to attach the polynucleotide probes and/or amplification product to a polymer matrix. Non-limiting examples of modification and polymer matrix that can be employed in accordance with the provided instances comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some embodiments, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some instances, the provided methods involve embedding the one or more polynucleotide probe sets and/or the amplification products in the presence of hydrogel subunits to form one or more hydrogel-embedded amplification products. In some instances, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some instances, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some instances, the RCA template may comprise the target analyte, or a part thereof, where the target analyte is a nucleic acid, or it may be provided or generated as a proxy, or a marker, for the analyte. In some instances, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some instances, a product herein includes a molecule or a complex generated in a series of reactions, e.g., hybridization, ligation, extension, replication, transcription/reverse transcription, and/or amplification (e.g., rolling circle amplification), in any suitable combination.

### Fluorescence Detection and Imaging

### Fluorescence Detection

Sequencing methods provided herein include detection of fluorescent nucleotides. Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some instances, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of fluorescent labels and nucleotides and/or polynucleotides conjugated to such fluorescent labels comprise those described elsewhere herein and those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some instances, non-limiting examples of techniques and methods applicable to the provided embodiments comprise those described in, for example, U.S. Pat. Nos. 4,757,141, 5,151,507 and 5,091,519. In some instances, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. In some instances, a fluorescent label comprises a signaling moiety that conveys information through the fluorescence absorption and/or emission properties of one or more molecules. Non-limiting examples of fluorescence properties comprise fluorescence intensity, fluorescence lifetime, emission spectrum characteristics and energy transfer.

### Imaging

In some embodiments, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some instances, fluorescence microscopy is used for detection and imaging of the sample. In some embodiments, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be or comprise any microscope that uses fluorescence to generate an image, whether it is a more simple set up like an epifluorescence microscope, or a more complicated design such as a confocal microscope, which uses optical sectioning to achieve better z-axis resolution of the sample to be imaged.

In some instances, confocal microscopy is used for detection and imaging of the sample. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immune-stained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some instances, a method herein comprises subjecting the sample to expansion microscopy methods and techniques. Expansion allows individual targets (e.g., mRNA or RNA transcripts) which are densely packed within a cell, to be resolved spatially in a high-throughput manner. Expansion microscopy techniques can be performed as described in, e.g., US 2016/0116384 and Chen et al., Science, 347, 543 (2015), each of which are incorporated herein by reference in their entirety. In some instances, the method does not comprise subjecting the sample to expansion microscopy. In some instances, the method does not comprise dissociating a cell from the sample such as a tissue or the cellular microenvironment. In some instances, the method does not comprise lysing the sample or cells therein. In some instances, the method does not comprise embedding the sample or molecules from the sample in an exogenous matrix.

In some cases, analysis is performed on one or more images captured, and may comprise processing the image(s) and/or quantifying signals observed. In some instances, images of signals from different fluorescent channels and/or nucleotide incorporation cycles can be compared and analyzed. In some instances, images of signals (or absence thereof) at a particular location in a sample from different fluorescent channels and/or sequential incorporation cycles can be aligned to analyze an analyte at the location. For instance, a particular location in a sample can be tracked and signal spots from sequential incorporation cycles can be analyzed to detect a target polynucleotide sequence (e.g., a barcode sequence or subsequence thereof) in an analyte at the location. The analysis may comprise processing information of one or more cell types, one or more types of analytes, a number or level of analyte, and/or a number or level of cells detected in a particular region of the sample. In some instances, the analysis comprises detecting a sequence e.g., a barcode sequence present in an amplification product at a location in the sample. In some instances, the number of signals detected in a unit area in the biological sample is quantified. In some instances, the signals detected at a corresponding position in the biological sample in a plurality of images taken at different z positions (e.g., in the depth direction) is quantified and analyzed.

### Detection and Analysis of RCPs

In some aspects, the sequencing methods disclosed herein include sequencing RCA products (RCPs) immobilized on a substrate. In some aspects, at least a subset of RCPs on the substrate are generated using a gapfill step, and the method includes sequencing RCPs that were generated using the gapfill step. In some embodiments, the method further includes comprise hybridization-based detection of at least a subset of the RCPs immobilized on the substrate. In some cases, the sequencing methods include imaging the biological sample (e.g., on a different substrate prior to transfer of the target nucleic acids to the substrate comprising capture agents, or on the same substrate prior to removal of the biological sample) to detect fluorescent nucleotides, including a nucleotide with modifiable fluorescence. In some embodiments, the method comprises overlaying or superimposing data obtained from imaging the biological sample with data obtained from detecting the rolling circle amplification products on the substrate. For example, in some cases tissue segmentation information (e.g., based on H&E staining or immunofluorescence) is overlaid with the detected RCPs to correlate spatial localizations of RCPs on the substrate with the position of the corresponding analytes in the biological sample. In some cases, one or more fiducial markers are used to help correlate the positions in the biological sample with positions of RCPs on the substrate. In some embodiments, a plurality of images of the biological sample is acquired. For examples, one or more images of the biological sample stained with a nuclear stain, a histological stain, and/or an immunologic stain can be acquired and one or more images can be acquired to detect RCPs in the biological sample. In some aspects, the plurality of images can be acquired separately.

### Barcode Sequences

In some embodiments, the sequencing methods described herein include sequencing a barcode. In some instances, an analyte described herein can be associated with one or more barcode(s), e.g., at least two, three, four, five, six, seven, eight, nine, ten, or more barcodes. Barcodes can be used to spatially-resolve molecular components found in biological samples, for example, within a cell or a tissue sample. A barcode can be attached to an analyte or to another moiety or structure (e.g., a target-specific antibody) in a reversible or irreversible manner. In some embodiments, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode includes two or more sub-barcodes (or barcode segments) that together function as a single barcode. For example, a polynucleotide barcode can include two or more polynucleotide sequences (e.g., sub-barcodes) that are contiguous or that are separated by one or more non-barcode sequences. In some instances, a barcode may comprise about 1, 2, 3, 4, 5, 6, 7, 8, 9, 10, or more than 10 sub-barcodes (or barcode segments). In some instances, each sub-barcode (or barcode segment) may comprise about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some instances, each non-barcode sequence may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding instances, the methods provided herein can include analyzing the barcodes by performing sequential hybridization and detection with a plurality of labelled probes (e.g., detection oligos) or by performing in situ sequencing.

In some instances, e.g., in a barcode sequencing method, barcode sequences are detected for identification of other molecules including nucleic acid molecules (DNA or RNA) that are longer than the barcode sequences themselves, as opposed to direct sequencing of the longer nucleic acid molecules. In some instances, an N-mer barcode sequence can comprise up to 4^{N} unique sequences given a sequencing read of N bases, and a much shorter sequencing read may be required for molecular identification compared to non-barcoded sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some instances, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

### VI. Cyclic Array Sequencing in a Flow Cell

In some instances, a method of sequencing as provided herein includes “cyclic array sequencing” of amplified template nucleic acid molecules. Cyclic array flow cell sequencing methods generally involve performing multiple cycles of an enzymatic reaction on an array of spatially-separated oligonucleotide features (e.g., clonally-amplified colonies of template nucleic acid fragments tethered to a support surface, e.g., a flow cell surface). In some instances, the template nucleic acid is modified with known adapter sequence(s) comprising, e.g., amplification and/or sequencing primer binding sites, and then affixed to the support surface (e.g., the lumen surface(s) of a flow cell) in a random or patterned array by hybridization to surface-tethered complementary capture probes (complementary to adapter sequences) on the support surface, clonally amplified, and then probed using the aforementioned sequencing reaction as described herein. In some embodiments, the flow cell sequencing comprises massively parallel sequencing reaction, whereby each enzymatic reaction cycle is used to query only one base (the “interrogation” nucleobase) of the template nucleic acid fragment in each oligonucleotide feature, but thousands to billions of template nucleic acid molecules may be processed in parallel. Performing repeated cycles is then used to progressively identify the nucleic acid sequence of each template nucleic acid molecule based on patterns of detection of a signal or detection of an absence of a signal associated with binding of a nucleotide comprising a modifiable fluorophore to the template, as detected over the course of multiple reaction cycles. In some embodiments, detection is often based on the use of modifiable fluorophores.

### Nucleic Acid Extraction

Nucleic acid extraction from cells or other biological samples may be performed using any of a variety of techniques. For example, a typical DNA extraction procedure may comprise: (i) collection of a cell or tissue sample from which DNA is to be extracted, (ii) disruption of cell membranes (i.e., cell lysis) to release DNA and other cytoplasmic components, (iii) treatment of the lysed sample with a concentrated salt solution to precipitate proteins, lipids, and RNA, followed by centrifugation to separate out the precipitated proteins, lipids, and RNA, and (iv) purification of DNA from the supernatant (e.g., using spin columns or paramagnetic beads) to remove detergents, proteins, salts, or other reagents used during the cell membrane lysis step. Non-limiting examples of methods for performing nucleic acid (e.g., DNA and RNA) extraction are described in, for example, Ali et al. (2017) “Current Nucleic Acid Extraction Methods and Their Implications to Point-of-Care Diagnostics”, BioMed Research International 2017:9306564, and Dairawan et al. (2020), “The Evolution of DNA Extraction Methods”, Am J Biomed Sci & Res 8(1):39-45, the entire contents of each of which are incorporated herein by reference.

A variety of suitable commercial nucleic acid extraction and purification kits are consistent with the disclosure herein. Examples include, but are not limited to, the QIAamp® kits (for isolation of genomic DNA from human samples) and DNAeasy kits (for isolation of genomic DNA from animal or plant samples) from Qiagen (Germantown, Md.), or the Maxwell® and ReliaPrep™ series of kits from Promega (Madison, Wis.).

### Sequencing Library Preparation

Sequence library preparation may be performed using any of a variety of techniques. Library preparation typically comprises performing the steps of, e.g., end repair, tailing, and ligation of adapter sequences to template nucleic acid fragments. Extracted nucleic acid molecules (e.g., DNA molecule), or fragments thereof, that are typically used as the input for sequencing library preparation often have overhangs containing single-stranded DNA (ssDNA overhangs), breaks in the phosphodiester backbone that exist on just one strand (nicks), and/or ssDNA regions internal to the duplex molecule (ssDNA gaps). End repair reactions (using, e.g., a combination of 3′ exonuclease digestion to remove 3′ overhangs and a strand displacing polymerase reaction using dNTPs to fill nicks and gaps) are used to correct these defects in order to maximize the yield for capturing and sequencing the extracted DNA, and result in the generation of blunt-ended, double-stranded DNA (dsDNA) molecules.

Tailing (e.g., A tailing) is an enzymatic method (using, e.g., a Taq DNA polymerase) for adding a non-templated nucleotide (e.g., an A nucleotide) to the 3′ end of a blunt-ended, double-stranded DNA molecule that facilitates the ligation of the adapter sequences used for sequencing.

One or more adapter sequences may then be ligated to the ends of the end-repaired and tailed template nucleic acid molecules. The adapter sequences may comprise, for example, (i) capture sequences (e.g., the Illumina p5 and p7 adapter sequences) that allow the nucleic acid molecules of the library to bind to a flow cell surface comprising complementary capture probes, (ii) amplification primer binding sites for use in performing reverse transcription and/or for generating clonally-amplified clusters on a flow cell surface, (iii) sequencing primer binding sites (e.g., the Illumina Rd1 and Rd2 sequencing primer binding site sequences) used to initiate sequencing. In addition to amplification and/or sequencing primer binding sites, in some instances the adapters may comprise a barcode sequence, e.g., a sample identification barcode sequence (such as the Illumina Index 1 and Index 2 sample identifier sequences).

Non-limiting examples of methods for performing sequencing library preparation are described in, for example, Head et al. (2014), “Library construction for next-generation sequencing: Overviews and challenges”, BioTechniques 56(1):61-77, and Hess et al. (2020), “Library preparation for next generation sequencing: A review of automation strategies”, Biotechnology Advances 41:107537, the entire contents of each of which are incorporated herein by reference.

### Nucleic Acid Amplification (Including Clonal Amplification)

In some instances, the disclosed methods for performing nucleic acid sequencing (e.g., in vitro and/or flow cell sequencing) may comprise performing one or more steps (e.g., 1, 2, 3, 4, 5, or more than 5) steps of nucleic acid amplification. Amplification reactions with respect to in situ based sequencing methods as described herein are discussed previously. In some instances, for example, one or more steps of nucleic acid amplification may be performed as part of sequencing library preparation and/or following sequencing library preparation. In some instances, one or more steps of nucleic acid amplification (e.g., using a solid-phase amplification technique such as bridge amplification) may be performed after the template molecules of a sequencing library have been tethered to a support surface (e.g., a flow cell surface) to generate clonally-amplified colonies of the tethered template nucleic acid fragments.

In some instances, nucleic acid amplification may be performed to amplify all of the nucleic acid molecules extracted from a biological sample (e.g., using a random primer sequence). In some instances, nucleic acid amplification may be performed to amplify a selected subset of nucleic acid molecules extracted from a biological sample (e.g., using one or more primer sequences designed to hybridize to portions of the sequences for one or more target nucleic acid molecules of interest, or to sequences adjacent thereto).

In some instances, nucleic acid amplification may be performed to amplify an entire sequencing library (e.g., using a primer sequence that hybridizes to a common amplification primer binding site in the sequencing library adapters). In some instances, nucleic acid amplification may be performed to amplify selected portions of the sequencing library (e.g., using one or more primer sequences designed to hybridize to one or more amplification primer binding sites associated with one or more identifier sequences (or barcodes) included in the sequencing library adapters).

Nucleic acid amplification may be performed using any of a variety of nucleic acid amplification techniques, including both thermal and/or isothermal nucleic acid amplification techniques. Examples of suitable thermal nucleic acid amplification techniques include, but are not limited to, polymerase chain reaction (PCR), multiplexed PCR, nested PCR, bridge PCR, reverse transcription PCR (RT-PCR). Examples of suitable isothermal nucleic acid amplification techniques include, but are not limited to, rolling circle amplification (RCA), nucleic acid sequence-based amplification (NASBA), loop-mediated isothermal amplification (LAMP), strand displacement amplification (SDA), helicase-dependent amplification (HDA), nicking enzyme amplification reaction (NEAR), and recombinase polymerase amplification (RPA). Examples of methods for performing nucleic acid amplification are described in, for example, Gill et al. (2008), “Nucleic Acid Isothermal Amplification Technologies—A Review”, Nucleosides, Nucleotides, and Nucleic Acids 27:224-243, Fakruddin et al. (2013), “Nucleic acid amplification: Alternative method of polymerase chain reaction”, J Pharm Bioallied Sci. 5(4): 245-252, and U.S. Pat. No. 8,143,008, the entire contents of each of which are incorporated herein by reference.

### Nucleotides

In some instances, the disclosed methods for performing nucleic acid sequencing (e.g., in situ and/or flow cell sequencing) may comprise the use of modified versions (e.g., comprising a modifiable fluorophore, as described elsewhere herein) of any of a variety of naturally-occurring nucleotides and/or functional analogs thereof (e.g., nucleotide analogs capable of hybridizing to a nucleic acid sequence in a sequence-specific/correctly base-paired manner) to probe a template nucleic acid sequence. Naturally-occurring nucleotides include deoxyribonucleotides (found in DNA) that comprise a deoxyribose sugar moiety, and ribonucleotides (found in RNA) that comprise a ribose sugar moiety. Naturally-occurring deoxyribonucleotides comprise a nucleobase (or “base”) selected from the group consisting of adenine (A), thymine (T), cytosine (C), or guanine (G). Naturally-occurring ribonucleotides comprise a nucleobase selected from the group consisting of uracil (U), adenine (A), cytosine (C), or guanine (G). In some instances, the nucleotides may be terminated (e.g., reversibly terminated). In some instances, the nucleotides may be conjugated to a detectable label, e.g., a fluorophore. In some instances, the nucleotides may be conjugated to a modifiable detectable label, e.g., a fluorophore comprising a cleavable quencher, pH-sensitive fluorophore. In some instances, the nucleotides may be conjugated to other moieties, e.g., reactive functional groups.

### Primers (or Primer Sequences)

In some instances, the disclosed methods for performing nucleic acid sequencing (e.g., in situ and/or flow cell sequencing) can comprise the use of primer sequences that are complementary to, e.g., a subsequence (or primer binding site) that is part of an endogenous nucleic acid target sequence or a sequence (or primer binding site) that is located at or near a barcode (identifier) sequence associated with a target analyte. In some instances, a primer sequence may be designed to hybridize to a primer binding site associated with a single target analyte sequence and/or an associated target-specific barcode sequence. In some instances, a primer sequence may be designed to hybridize to a sequence (or primer binding site) that is associated with a plurality of target analyte sequences and/or associated target-specific barcode sequences (e.g., at least 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, or more than 1000 target analyte sequences and/or associated target-specific barcode sequences).

### Polymerases

In some instances, the disclosed methods for performing nucleic acid sequencing (e.g., in situ and/or flow cell sequencing) may comprise performing one or more steps of nucleic acid amplification or replication using one or more polymerases. Examples of polymerases that may be used for amplification include, but are not limited to, DNA polymerases (e.g., Taq DNA polymerase), RNA polymerases, and/or reverse transcriptases.

As noted elsewhere herein, non-limiting examples of polymerases for use in rolling circle amplification (RCA) comprise DNA polymerases such phi29 (φ29) polymerase, Klenow fragment, Bacillus stearothermophilus DNA polymerase (BST), T4 DNA polymerase, T7 DNA polymerase, or DNA polymerase I. In some embodiments, DNA polymerases that have been engineered or mutated to have desirable characteristics can be employed. In some embodiments, the polymerase is phi29 DNA polymerase.

### Base-Calling

As noted elsewhere herein, the disclosed methods for performing nucleic acid sequencing (e.g., in situ and/or flow cell sequencing) may comprise inferring the sequence of a template nucleic acid molecule from a series of optical signals (e.g., fluorescence signals) detected in images acquired during a repetitive series of sequencing reaction cycles in a process referred to as “base-calling”. The interplay of sequencing chemistry, opto-fluidics hardware, optical sensors, and signal processing software utilized in sequencing platforms affects the types of errors made during sequencing (see, e.g., Lederberger et al. (2011), “Base-calling for next-generation sequencing platforms”, Brief Bioinform. 12(5): 489-497). The characterization of errors associated with the sequencing process and implementation of chemistry-, imaging-, and/or signal processing software-based methods for minimizing sequence errors are thus important for maximizing the accuracy of sequencing results.

In four-color sequencing-by-synthesis methods, for example, a set of four images—one image for each of four detection channels corresponding to the emission wavelengths for four fluorophores used to label the reversibly terminated nucleotides—are acquired in each sequencing cycle. Processing of the images to detect fluorescence intensity signals produces an intensity quadruple for the location of each sequencing colony on a flow cell surface (or the location of each target analyte, or amplified representation thereof (e.g., an RCP) in the case of in situ sequencing), where each value represents the intensity of the fluorescence signal for the detection channels corresponding to A, C, G and T. Ideally, the channel in which the maximum intensity occurs would be the base that is “called” for a given RCP or sequencing colony (or target analyte) in a given cycle. However, the chemical processes involved in sequencing are imperfect, leading to errors in base-calling (see, e.g., Cacho, et al. (2016), “A Comparison of Base-calling Algorithms for Illumina Sequencing Technology”, Briefings in Bioinformatics 17(5):786-795). In some sequencing-by-synthesis (SBS) platforms, for example, sources of error may include phasing (or lagging; e.g., where the primed template nucleic acid molecules at one or more locations fail to incorporate the next base due to variation in polymerase reaction kinetics), pre-phasing (or leading; e.g., where more than one nucleotide is incorporated in a single cycle due to, e.g., impurities in the reversibly terminated nucleotides), signal decay (due to, e.g., photobleaching and/or loss of template nucleic acid during the sequencing process), and cross-talk (e.g., when two or more fluorophore emission spectra overlap, which may cause a positive correlation between signal intensities measured in the corresponding detection channels).

A variety of statistical approaches have been developed to correct for, or minimize, such errors and generate more accurate base-calls. Examples include, but are not limited to, AYB (Goldman Group, European Molecular Biology Laboratory—European Bioinformatics Institute, Cambridgeshire, UK), and Bustard (Illumina, Inc., San Diego, CA).

### Sequence Reads

The output of the base-calling process applied to optical signals detected in a series of images of a biological sample or flow cell surface acquired during a cycling sequencing process consists of a plurality of sequence reads, e.g., the nucleotide sequences determined for all or a portion of a template nucleic acid molecule (e.g., an endogenous nucleic acid analyte or a barcode sequence associated with a target analyte).

In some instances, the sequence reads generated using the disclosed methods for in situ and/or flow cell sequencing may comprise sequence reads of at least about 10, 20, 30, 40, 50, 60, 70, 80, 90, 100 or more nucleotides or base pairs of the template nucleic acid sequences. In some instances, the sequence reads generated using the disclosed methods may comprise sequence reads of at least about 100, 120, 140, 160, 180, 200, 220, 240, 260, 280, 300, 320, 340, 360, 380, 400, or more than 400 nucleotides or base pairs of the template nucleic acid sequences.

In some instances, the disclosed methods for in situ or flow cell sequencing may generate at least about 100, 200, 300, 400, 500, 600, 700, 800, 900, 1,000, or more sequencing reads per run. In some instances, the disclosed method may generate at least about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 10⁶, 5×10⁶, 10⁷, or more than 10⁷ sequencing reads per run.

### Sequence Assembly & Alignment

In some instances, the disclosed methods for in situ and/or flow cell sequencing may comprise assembly of longer template nucleic acid sequences, e.g., genome fragments or whole genomes, from a plurality of relatively short sequence reads. Sequence assembly may be performed by identifying the overlapping sequences from multiple short sequence reads to assemble longer, contiguous sections of sequence.

In some instances, the disclosed methods for in situ and/or flow cell sequencing may comprise identifying a code word corresponding to a sequence read or an assembled sequence, where the code word is one of a plurality of code words in a codebook that includes assignment of each of the plurality of code words to a target analyte of interest. The sequence read or assembled sequence may thus be used to identify a specific target analyte (based on the corresponding code word) in, e.g., a multiplexed in situ detection or sequencing assay.

In some instances, the disclosed methods for in situ and/or flow cell sequencing may comprise alignment of sequence reads and/or assembled sequences to a known reference sequence or consensus sequence (e.g., the GRCh38 human reference genome (Genome Reference Consortium)) from the same or a similar organism. Alignment to a reference sequence or consensus sequence may be used to identify gaps, errors, or variants in the assembled sequence. Any of a variety of bioinformatics software programs may be used to assemble longer sequences from relatively short sequence reads. Examples include, but are not limited to, DBG2OLC (see, e.g., Ye et al. (2016), “DBG2OLC: Efficient Assembly of Large Genomes Using Long Erroneous Reads of the Third Generation Sequencing Technologies”, Scientific Reports 6:31900), SPAdes (see, e.g., Bankevich et al. (2012), “SPAdes: A New Genome Assembly Algorithm and Its Applications to Single-Cell Sequencing”, J. Computational Biol. 19(5):455-477), SparseAssembler (see, e.g., Ye et al. (2012), “Exploiting Sparseness in de novo Genome Assembly”, BMC Bioinformatics 13(Suppl 6):S1), Fermi (see, e.g., Li (2012), “Exploring Single-Sample SNP and INDEL Calling with Whole-Genome de novo Assembly”, Bioinformatics 28(14):1838-1844), and String Graph Assembler (SGA) (see, e.g., Simpson et al. (2012), “Efficient de novo Assembly of Large Genomes Using Compressed Data Structures”, Genome Res. 22: 549-556).

### VII. Opto-Fluidic Instrument

In some instances, the sequencing methods described herein (e.g., in situ sequence or flow cell sequencing) include using instruments having integrated optics and fluidics modules (“opto-fluidic instruments” or “opto-fluidic systems”) for detecting target molecules (e.g., nucleic acids, proteins, antibodies, etc.) in biological samples (e.g., one or more cells or a tissue sample) as described herein.

In an opto-fluidic instrument, the fluidics module is configured to deliver one or more reagents (e.g., compositions comprising nucleotides, primers, detectably-labeled probes and/or non-labeled probes, polymerases and/or other enzymes, deprotection reagents, buffers, etc.) to the biological sample (e.g., to a sample cartridge within which the biological sample is contained) or to a flow cell (e.g., within which nucleic acid molecules extracted from the biological sample have been tethered) and/or to remove spent reagents therefrom. In some instances, one or more sample preparation steps (e.g., fixing, embedding, sample clearing, and/or nucleic acid extraction (in the case that nucleic acid molecules are to be extracted and sequenced in a flow cell)) may be performed prior to the sample being placed on the instrument. In some instances, the fluidics module is configured to deliver one or more further reagents (e.g., primary probe(s) such as circular probe(s) or circularizable probe(s) or probe set(s)) and/or to remove non-specifically hybridized probe(s). In some instances, the fluidics module is configured to deliver one or more detectably labeled probes and optionally intermediate probes to detect the target analytes, or amplified representatives thereof (e.g., RCP(s)) in the biological sample. In some instances, the fluidics module is configured to deliver one or more composition (e.g., composition nucleotides, as well as primers, polymerases, deprotection reagents, etc.) to sequence, e.g., native nucleic acid sequences, barcode sequences associated with target analytes, or amplified copies thereof (e.g., barcode sequences included in RCP(s)) in the biological sample. In some instances, the fluidics module is configured to deliver one or more compositions (e.g., compositions of nucleotides, as well as primers, polymerases, deprotection reagents, etc.) to a flow cell to sequence, e.g., native nucleic acid sequences, barcode sequences, or amplified copies thereof extracted from the biological sample.

Additionally, the optics module is configured to illuminate the biological sample (or flow cell) with light having one or more spectral emission curves (over a range of wavelengths) and subsequently capture one or more images of emitted light signals from the biological sample (or flow cell) during one or more decoding (e.g., probing or sequencing) cycles. In various instances, the captured images may be processed in real time and/or at a later time to determine the presence of the one or more target molecules in the biological sample, as well as two-dimensional and/or three-dimensional position information associated with each detected target molecule within the biological sample. In various instances, the captured images of a flow cell surface are processed in real time and/or at a later time to determine the sequence of the one or more nucleic acid sequences (e.g., barcode sequences associated with one or more target molecules) that have been extracted from a biological sample. In some embodiment, the optics module further comprises an autofocus mechanism configured to maintain focus at a specified sample plane (e.g., a plane that is perpendicular to the optical axis of an objective lens of the optics module).

Additionally, the opto-fluidics instrument includes a sample module configured to receive (and, optionally, secure) one or more biological samples (e.g., biological samples contained with one or more sample cartridges), or to receive (and, optionally, secure) one or more flow cells. In some instances, the sample module includes an X-Y stage configured to move the biological sample (or flow cell) along an X-Y plane (e.g., perpendicular to the optical axis of an objective lens of the optics module).

In various instances, the opto-fluidic instrument is configured to analyze one or more target molecules (e.g., one or more target RNAs) in their naturally occurring place (i.e., in situ) within the biological sample. In some instances, the opto-fluidic instrument is configured to analyze one or more target RNAs in relative spatial locations within the biological sample. For example, an opto-fluidic instrument may be an in-situ analysis system used to analyze a biological sample and detect target molecules including, but not limited to, DNA, RNA, proteins, antibodies, and/or the like. In some instances, the in situ analysis system is used to detect one or more target RNAs using target-primed rolling circle amplification (RCA) according to the methods disclosed herein.

In various instances, the opto-fluidic instrument may be configured to perform in situ target molecule detection via base-by-base sequencing (e.g., by sequencing an identifier sequence such as a barcode sequence associated with a target molecule) and/or any imaging or target molecule detection technique. That is, for example, an opto-fluidic instrument may include a fluidics module that includes fluids needed for establishing the experimental conditions required for the probing or sequencing of target molecules (or associate barcode sequences) in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

### VIII. Compositions

In some embodiments, a composition comprising a first nucleotide comprising a first nucleobase type and a second fluorescent nucleotide comprising a second nucleobase type, wherein the first fluorescent nucleotide comprises a quencher, wherein the quencher comprises a cleavable moiety.

In some embodiments, a composition comprising a first nucleotide comprising a first nucleobase type and a second fluorescent nucleotide comprising a second nucleobase type, wherein the first fluorescent nucleotide comprises a quencher-attachment moiety wherein a quencher attaches.

In some embodiments, a composition of a first nucleotide comprising a first nucleobase type, a second fluorescent nucleotide comprising a second nucleobase type, wherein the first fluorescent nucleotide is pH-sensitive, emitting fluorescence in an acidic environment and not emitting fluorescence in a basic environment.

In some embodiments, a composition of a first nucleotide comprising a first nucleobase type, a second fluorescent nucleotide comprising a second nucleobase type, wherein the first fluorescent nucleotide is pH-sensitive, emitting fluorescence in a basic environment and not emitting fluorescence in an acidic environment.

In some embodiments, a composition of a first nucleotide comprising a first nucleobase type, a second fluorescent nucleotide comprising a second nucleobase type, wherein the first fluorescent nucleotide is reducing agent-sensitive, emitting fluorescence in a nonreducing environment and not emitting fluorescence in a reducing environment.

### IX. Kits and Systems

In some embodiments, provided herein are kits or systems for sequencing nucleic acid molecules, including kits or systems for sequencing and analysis of target nucleic acids in a biological sample according to any of the methods described herein.

In some aspects, provided herein are kits or systems for performing a cycle of a nucleic acid sequencing reaction comprising a first fluorescent nucleotide analogue or modified nucleotide, and a second nucleotide analogue or modified nucleotide in a fluorescent form. In some embodiments, the second nucleotide analogue or modified nucleotide is configured to be modifiable to generate a non-fluorescent form of the second nucleotide analogue or modified nucleotide. In some embodiments, the kit or system further comprises a quencher that is configured for attachment to the second nucleotide analogue or modified nucleotide. In some embodiments, the quencher is configured for attachment to the second nucleotide analogue or modified nucleotide using click chemistry.

In some aspects, provided herein are kits or systems for performing a cycle of a nucleic acid sequencing reaction comprising a first fluorescent nucleotide analogue or modified nucleotide, and a second nucleotide analogue or modified nucleotide in a fluorescent form. In some embodiments, the second nucleotide analogue or modified nucleotide is configured to be modifiable to generate a non-fluorescent form of the second nucleotide analogue or modified nucleotide. In some embodiments, the kit or system further comprises the second nucleotide analogue or modified nucleotide comprising a reducing agent-sensitive dye that can generate a non-fluorescent form of the second nucleotide analogue or modified nucleotide by exposure to a reducing agent. In some embodiments, the kit or system further comprises the reducing agent.

In some aspects, provided herein are kits or systems for performing a cycle of a nucleic acid sequencing reaction comprising a first fluorescent nucleotide analogue or modified nucleotide, and a second nucleotide analogue or modified nucleotide in a fluorescent form. In some embodiments, the second nucleotide analogue or modified nucleotide is configured to be modifiable to generate a non-fluorescent form of the second nucleotide analogue or modified nucleotide. In some embodiments, the kit or system further comprises the second nucleotide analogue or modified nucleotide comprising a sulfur-containing-sensitive dye that can generate a non-fluorescent form of the second nucleotide analogue or modified nucleotide by exposure to a reducing agent. In some embodiments, the kit or system further comprises the reducing agent.

In some aspects, provided herein is a kit or system comprising modifiable nucleotides or nucleotide analogues described herein. In some embodiments, the kit or system further comprises any of the primers described herein. In some embodiments, the kit or system further comprises any of the polymerases described herein. In some embodiments, the kit or system further comprises a quencher that is configured for attachment to the second nucleotide analogue or modified nucleotide described herein. In some embodiments, the kit or system further comprises the quencher configured for attachment to the nucleotide analogue or modified nucleotide using click chemistry. In some embodiments, the kit or system further comprises the quencher configured for attachment to the nucleotide analogue or modified nucleotide using an azide-DBCO reaction, a tetrazine-TCO reaction, or an azide-alkyne reaction. In some embodiments, the quencher is configured for attachment to the second nucleotide analogue or modified nucleotide using an azide-DBCO reaction, a tetrazine-TCO reaction, or an azide-alkyne reaction.

In some aspects, provided herein is a kit or system comprising modifiable nucleotides or nucleotide analogues described herein. In some embodiments, the kit or system further comprises any of the primers described herein. In some embodiments, the kit or system further comprises any of the polymerases described herein. In some embodiments, the kit or system further comprises modifiable nucleotides or nucleotide analogues comprising a reducing agent-sensitive dye described herein. In some embodiments, the kit or system further comprises the reducing agent-sensitive dye that is deactivatable by exposure to a reducing agent. In some embodiments, the kit or system further comprises the reducing agent.

In some aspects, provided herein is a kit or system comprising modifiable nucleotides or nucleotide analogues described herein. In some embodiments, the kit or system further comprises any of the primers described herein. In some embodiments, the kit or system further comprises any of the polymerases described herein. In some embodiments, the kit or system further comprises modifiable nucleotides or nucleotide analogues comprising a sulfur-containing-sensitive dye described herein. In some embodiments, the kit or system further comprises the sulfur-containing-sensitive dye that is deactivatable by exposure to a reducing agent. In some embodiments, the kit or system further comprises the reducing agent.

In some aspects, provided herein are kits or systems for performing a cycle of a nucleic acid sequencing reaction comprising a first fluorescent nucleotide analogue or modified nucleotide, and a second nucleotide analogue or modified nucleotide in a non-fluorescent form. In some embodiments, the second nucleotide analogue or modified nucleotide is configured to be modifiable to generate a fluorescent form of the second nucleotide analogue or modified nucleotide. In some embodiments, the second nucleotide analogue or modified nucleotide in the kit or system further comprises a cleavable quencher.

In some aspects, provided herein are kits or systems for performing a cycle of a nucleic acid sequencing reaction comprising a first fluorescent nucleotide analogue or modified nucleotide, and a second nucleotide analogue or modified nucleotide in a non-fluorescent form. In some embodiments, the second nucleotide analogue or modified nucleotide is configured to be modifiable to generate a fluorescent form of the second nucleotide analogue or modified nucleotide. In some embodiments, the kit or system further comprises the second nucleotide analogue or modified nucleotide in the non-fluorescent form comprising a pH-sensitive dye that is activatable by protonation.

In some aspects, provided herein is a kit or system for performing in situ sequencing comprising a mixture comprising a first fluorescent nucleotide analogue or modified nucleotide, and a second nucleotide analogue or modified nucleotide in a non-fluorescent form as described herein, and one or more further components for performing the in situ sequencing reaction. In some embodiments, the further components include an imaging buffer. In some embodiments, the imaging buffer comprises an oxygen scavenger that releases a proton. In some embodiments, the one or more further components include a polymerase, a primer, a support for a tissue or cell sample, or any combination thereof. In some embodiments, the kit or system further comprises any of the circular probes and/or circularizable probes or probe sets disclosed herein. In some embodiments, the kit or system comprises a polymerase for rolling circle amplification.

In some aspects, provided herein is a kit or system for performing in situ sequencing comprising a mixture comprising a first fluorescent nucleotide analogue or modified nucleotide, and a second nucleotide analogue or modified nucleotide in a fluorescent form as described herein, and one or more further components for performing the in situ sequencing reaction. In some embodiments, the further components include an imaging buffer. In some embodiments, the one or more further components include a polymerase, a primer, a support for a tissue or cell sample, or any combination thereof. In some embodiments, the kit or system further comprises any of the circular probes and/or circularizable probes or probe sets disclosed herein. In some embodiments, the kit or system comprises a polymerase for rolling circle amplification.

In some embodiments, provided herein is a kit or system for flow cell sequencing comprising a mixture comprising modifiable nucleotides or nucleotide analogues as described herein, and one or more further components for performing the flow cell sequencing reaction. In some embodiments, the further components include an imaging buffer. In some embodiments, the imaging buffer comprises an oxygen scavenger that releases a proton. In some embodiments, the one or more further components include a polymerase, a primer, a flow cell, primers, adapters for sequencing library preparation, or any combination thereof.

The various components of the kit or system may be present in separate containers or certain compatible components may be pre-combined into a single container. In some embodiments, the kits or systems further contain instructions for using the components of the kit or system to practice the provided methods. In some embodiments, each mixture of modifiable nucleotides or nucleotide analogues is provided in separate containers. In some embodiments, sets of modifiable nucleotides or nucleotide analogues (as described elsewhere) are provided together in a single container, such as a tube. In some embodiments, each modifiable nucleotide or nucleotide analogue is provided in separate containers. In some embodiments, a first fluorescent nucleotide analogue or modified nucleotide, is provided together in a first container, and a second nucleotide analogue or modified nucleotide in a non-fluorescent form is provided in a second container. In some embodiments, a first fluorescent nucleotide analogue or modified nucleotide, is provided together in a first container, and a second nucleotide analogue or modified nucleotide in a fluorescent form is provided in a second container.

In some embodiments, provided herein is a kit or system for sequencing a template nucleic acid molecule, comprising contacting the biological sample with a first set of nucleotides and a second set of nucleotides. In some embodiments, the first set of nucleotides comprises a non-fluorescent form of the nucleotide or nucleotide analogue and the second set of nucleotides comprises a fluorescent form of the nucleotide or nucleotide analogue. In some embodiments, the non-fluorescent nucleotides comprises a fluorophore comprising a quenched fluorophore. In some embodiments, the non-fluorescent fluorophore is modified after detecting the fluorescence of the second set of nucleotides. In some embodiments, the fluorophore is modified to generate the fluorescent form of the fluorophore. In some embodiments, the modifications comprise cleaving a quencher, cleaving a disulfide moiety associated with the quencher, and/or increasing protonation during the nucleic acid sequencing reaction. In some embodiments, after modifying the fluorescence of the first set of nucleotides, the fluorescence of the first set is detected. In some embodiments, the kit or system further comprises a third nucleotide. In some embodiments, the third nucleotide comprises a fluorophore that emits fluorescence detectable in a different channel than the first nucleotide analogue or modified nucleotide and the fluorescent form of the second nucleotide analogue or modified nucleotide. In some embodiments, the kit or system further comprises a fourth nucleotide. In some embodiments, the fourth nucleotide comprises a fluorophore that is detectable in a different channel than the fluorophore of the third nucleotide, the first nucleotide analogue or modified nucleotide, and the fluorescent form of the second nucleotide analogue or modified nucleotide. In some embodiments, the fourth nucleotide does not comprise a fluorescent dye.

In some embodiments, provided herein is a kit or system for sequencing a template nucleic acid molecule, comprising contacting the biological sample with a first set of nucleotides and a second set of nucleotides. In some embodiments, the first set of nucleotides comprises a fluorescent form of the nucleotide or nucleotide analogue and the second set of nucleotides comprises a fluorescent form of the nucleotide or nucleotide analogue. In some embodiments, the fluorescent nucleotides comprises a fluorophore comprising a quencher-attachment moiety. In some embodiments, the fluorescent fluorophore is modified after detecting the fluorescence of the first set of nucleotides. In some embodiments, the fluorophore is modified to generate the non-fluorescent form of the fluorophore. In some embodiments, the modifications comprise attaching a quencher, quenching fluorescence, and/or decreasing protonation during the nucleic acid sequencing reaction. In some embodiments, after modifying the fluorescence of the first set of nucleotides, the fluorescence of the second set is detected. In some embodiments, the kit or system further comprises a third nucleotide. In some embodiments, the third nucleotide comprises a fluorophore that emits fluorescence detectable in a different channel than the first nucleotide analogue or modified nucleotide and the fluorescent form of the second nucleotide analogue or modified nucleotide. In some embodiments, the kit or system further comprises a fourth nucleotide. In some embodiments, the fourth nucleotide comprises a fluorophore that is detectable in a different channel than the fluorophore of the third nucleotide, the first nucleotide analogue or modified nucleotide, and the fluorescent form of the second nucleotide analogue or modified nucleotide. In some embodiments, the fourth nucleotide does not comprise a fluorescent dye.

In some embodiments, the kits or systems can contain reagents and/or consumables required for performing one or more steps of the provided methods. In some embodiments, the kits or systems contain reagents for fixing, embedding, and/or permeabilizing the biological sample. In some embodiments, the kits or systems contain reagents, such as enzymes and buffers for ligation and/or amplification, such as ligases and/or polymerases. In some embodiments, the kit or system also comprises any of the reagents described herein, e.g., wash buffer and ligation buffer. In some embodiments, the kits or systems contain reagents for detection and/or sequencing, such as barcode detection probes or detectable labels. In some embodiments, the kits or systems optionally contain other components, for example nucleic acid primers.

### X. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### XI. Enumerated Embodiments

The disclosure further provides the following enumerated embodiments.

Embodiment 1. A method, comprising:
contacting a biological sample with a composition comprising a first fluorescent nucleotide analogue or modified nucleotide and a second nucleotide analogue or modified nucleotide in a fluorescent form; and
performing a cycle of a nucleic acid sequencing reaction on a template nucleic acid in the biological sample, wherein the nucleic acid sequencing reaction comprises, sequentially: a) detecting a fluorescent intensity of the second nucleotide analogue or modified nucleotide, wherein after detecting the fluorescent intensity of the second dye, the second nucleotide analogue or modified nucleotide is modified to generate a non-fluorescent form of the second nucleotide analogue or modified nucleotide, and b) detecting a fluorescent intensity of the first fluorescent nucleotide analogue or modified nucleotide.

Embodiment 2. The method of embodiment 1, wherein the non-fluorescent form of the second nucleotide analogue or modified nucleotide comprises a quencher.

Embodiment 3. The method of embodiment 2, wherein the second nucleotide analogue or modified nucleotide is modified to generate the non-fluorescent form of the second nucleotide analogue or modified nucleotide by attaching the quencher to the second nucleotide analogue or modified nucleotide.

Embodiment 4. The method of embodiment 3, wherein the quencher is attached to the second nucleotide analogue or modified nucleotide using click chemistry.

Embodiment 5. The method of embodiment 3 or embodiment 4, wherein the quencher is attached to the second nucleotide analogue or modified nucleotide using an azide-DBCO reaction, a tetrazine-TCO reaction, or an azide-alkyne reaction.

Embodiment 6. The method of any one of embodiments 1-5, wherein the second nucleotide analogue or modified nucleotide in the fluorescent form is a fluorescent nucleotide analogue.

Embodiment 7. The method of embodiment 6, wherein the fluorescent nucleotide analogue is a pteridine fluorescent nucleotide analogue.

Embodiment 8. The method of any one of embodiments 1-5, wherein the second nucleotide analogue or modified nucleotide in the fluorescent form comprises a fluorophore.

Embodiment 9. The method of any one of embodiments 1-5, wherein the second nucleotide analogue or modified nucleotide comprises a pH-sensitive dye.

Embodiment 10. The method of embodiment 9, wherein the second nucleotide analogue or modified nucleotide is modified to generate the non-fluorescent form of the second nucleotide analogue or modified nucleotide by deprotonation of the second nucleotide analogue or modified nucleotide.

Embodiment 11. The method of any one of embodiments 1-5, wherein the second nucleotide analogue or modified nucleotide comprises a reducing agent-sensitive dye comprising a disulfide moiety, wherein the disulfide moiety is cleavable upon contact with a reducing agent, and wherein the reducing agent-sensitive dye becomes non-fluorescent when the disulfide moiety is cleaved, optionally wherein the reducing agent-sensitive dye is selected from the group consisting of:

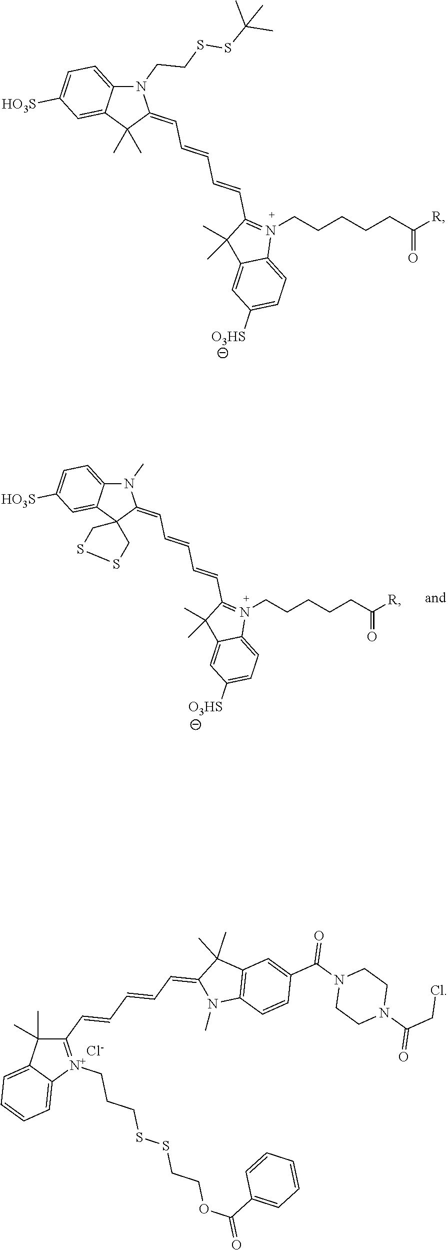

Embodiment 12 The method of embodiment 11, wherein the second nucleotide analogue or modified nucleotide is modified to generate the non-fluorescent form of the second nucleotide analogue or modified nucleotide by contacting the second nucleotide analogue or modified nucleotide with the reducing agent.

Embodiment 13. A method, comprising:
contacting a biological sample with a composition comprising a first fluorescent nucleotide analogue or modified nucleotide and a second nucleotide analogue or modified nucleotide, wherein the second nucleotide analogue or modified nucleotide is in a non-fluorescent form; and
performing a cycle of a nucleic acid sequencing reaction comprising a template nucleic acid in the biological sample, wherein the nucleic acid sequencing reaction comprises, sequentially: a) detecting a fluorescent intensity of the first fluorescent nucleotide analogue or modified nucleotide, wherein after detecting the first fluorescent nucleotide analogue or modified nucleotide, the second nucleotide analogue or modified nucleotide is modified to generate a fluorescent form of the second nucleotide analogue or modified nucleotide, and b) detecting a fluorescent intensity of the second nucleotide analogue or modified nucleotide.

Embodiment 14. The method of embodiment 13, wherein the non-fluorescent form of the second nucleotide analogue or modified nucleotide comprises a quencher.

Embodiment 15. The method of embodiment 14, wherein the second nucleotide analogue or modified nucleotide is modified to generate a fluorescent form of the second nucleotide analogue or modified nucleotide by cleaving the quencher.

Embodiment 16. The method of embodiment 14 or embodiment 15, wherein the non-fluorescent form of the second nucleotide analogue or modified nucleotide comprises a fluorophore, and wherein the quencher is capable of quenching the fluorophore.

Embodiment 17. The method of any one of embodiments 14-16, wherein the non-fluorescent form of the second nucleotide analogue or modified nucleotide comprises a quencher linked to the second nucleotide analogue or modified nucleotide via a cleavable linker.

Embodiment 18. The method of embodiment 17, wherein the non-fluorescent form of the second nucleotide analogue or modified nucleotide comprises a quencher linked to the second nucleotide analogue or modified nucleotide via a reduction-cleavable disulfide bond.

Embodiment 19. The method of any one of embodiments 13-18, wherein the second nucleotide analogue or modified nucleotide in its fluorescent form is a fluorescent nucleotide analogue.

Embodiment 20. The method of embodiment 19, wherein the fluorescent nucleotide analogue is a pteridine fluorescent nucleotide analogue.

Embodiment 21. The method of any one of embodiments 1-20, wherein the first nucleotide analogue or modified nucleotide and the fluorescent form of the second nucleotide analogue or modified nucleotide emit fluorescence with distinguishable wavelengths.

Embodiment 22. The method of embodiment 13, wherein the second nucleotide analogue or modified nucleotide comprises a pH-sensitive dye.

Embodiment 23. The method of embodiment 22, wherein the second nucleotide analogue or modified nucleotide is modified to generate the fluorescent form of the second nucleotide analogue or modified nucleotide by protonation of the second nucleotide analogue or modified nucleotide.

Embodiment 24. The method of embodiment 22 or embodiment 23, wherein the pH-sensitive fluorescent dye is a rhodamine derivative, wherein the pH-sensitive fluorescent dye has the following structure under acidic conditions:

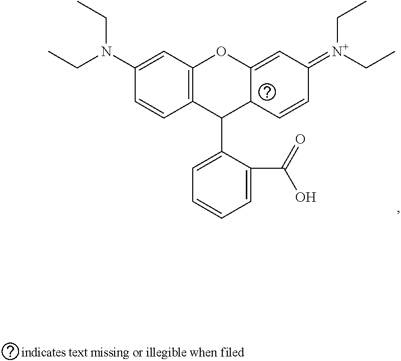 [SMILES: CCN(CC)c1ccc2c(c1)OC1=CC(=[N+](CC)CC)C=CC1C2c1ccccc1C(=O)O]

and the following structure under basic conditions:

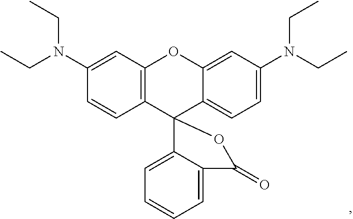 [SMILES: CCN(CC)c1ccc2c(c1)Oc1cc(N(CC)CC)ccc1C21OC(=O)c2ccccc21]

wherein the pH-sensitive dye is fluorescent in acidic conditions and non-fluorescent in basic conditions.

Embodiment 25. The method of any one of embodiments 22-24, wherein an imaging buffer is used during the nucleic acid sequencing reaction, wherein the imaging buffer comprises an oxygen scavenger that releases a proton.

Embodiment 26. The method of embodiment 25, wherein the pH-sensitive dye has a pKA, and in a) the imaging buffer has a pH that is above the pKA of the pH-sensitive dye, and wherein in b) the imaging buffer has a pH that is below the pKA of the pH-sensitive dye.

Embodiment 27. The method of embodiment 25 or embodiment 26, wherein the pH of the imaging buffer decreases during the nucleic acid sequencing reaction.

Embodiment 28. The method of any one of embodiments 1-27, wherein the method comprises contacting the sample with a third nucleotide.

Embodiment 29. The method of embodiment 28, wherein the third nucleotide comprises a fluorescent dye that emits fluorescence detectable in a different channel than the first nucleotide analogue or modified nucleotide and the fluorescent form of the second nucleotide analogue or modified nucleotide.

Embodiment 30. The method of embodiment 28 or 29, wherein the method further comprises contacting the sample with a fourth nucleotide.

Embodiment 31. The method of embodiment 30, wherein the fourth nucleotide comprises a fluorescent dye that is detectable in a different channel than the fluorescent dye of the third nucleotide, the first nucleotide analogue or modified nucleotide, and the fluorescent form of the second nucleotide analogue or modified nucleotide.

Embodiment 32. The method of embodiment 30, wherein the fourth nucleotide does not comprise a fluorescent dye.

Embodiment 33. The method of any one of embodiments 30-32, wherein the biological sample is contacted with a nucleotide mixture comprising the first nucleotide, the second nucleotide, the third nucleotide, and the fourth nucleotide.

Embodiment 34. The method of any one of embodiments 1-33, wherein the method comprises washing the sample to remove unbound nucleotides prior to the detecting in b) and/or prior to the detecting in c).

Embodiment 35. The method of any one of embodiments 1-34, wherein the nucleic acid sequencing reaction is a sequencing-by-synthesis reaction or a sequencing-by-binding reaction.

Embodiment 36. The method of any one of embodiments 1-35, comprising performing an additional cycle of a nucleic acid sequencing reaction to identify at least one additional base of the template nucleic acid.

Embodiment 37. The method of embodiment 36, wherein the at least one additional cycle comprises at least 2, 5, 10, 20, 30, 40, or 50 additional cycles.

Embodiment 38. The method of any one of embodiments 1-37, wherein the first nucleotide, second nucleotide, third nucleotide, and fourth nucleotide comprise different nucleobases selected from the group consisting of A, T or U, C, and G.

Embodiment 39. The method of embodiment 38, wherein the first nucleotide, second nucleotide, third nucleotide, and fourth nucleotide are reversibly terminated nucleotides.

Embodiment 40. The method of any one of embodiments 1-39, wherein the template nucleic acid comprises a DNA molecule.

Embodiment 41. The method of any one of embodiments 1-39, wherein the template nucleic acid comprises an RNA molecule.

Embodiment 42. The method of any one of embodiments 1-41, wherein the template nucleic acid comprises a target analyte nucleic acid sequence.

Embodiment 43. The method of any one of embodiments 1-40, wherein the template nucleic acid comprises a barcode sequence associated with an analyte in the biological sample.

Embodiment 44. The method of any one of embodiments 1-43, wherein the prior to the nucleic acid sequencing reaction, a circularizable probe hybridizes to a target analyte in the biological sample or to a labeling agent bound to the target analyte and is ligated to form a circularized probe, wherein the method further comprises performing rolling circle amplification of the circularized probe to generate the template nucleic acid.

Embodiment 45. The method of any one of embodiments 1-44, wherein the template nucleic acid is a rolling circle amplification product (RCP).

Embodiment 46. The method of embodiment 44 or 45, wherein the circularizable probe is a padlock probe sequence.

Embodiment 47. The method of embodiment 46, wherein the probe sequence comprises one or more barcode region.

Embodiment 48. The method of embodiment 44, wherein the target analyte comprises an mRNA molecule.

Embodiment 49. The method of any one of embodiments 1-48, wherein the template nucleic acid to be sequenced is attached to a solid support.

Embodiment 50. The method of embodiment 49, wherein the solid support comprises a sequencing flow cell.

Embodiment 51. The method of any one of embodiments 1-50, wherein the template nucleic acid is a first template nucleic acid in the biological sample, and wherein the method comprises performing the cycle of the nucleic acid sequencing reaction on a second template nucleic acid in the biological sample.

Embodiment 52. The method of embodiment 51, wherein the first template nucleic acid comprises a nucleotide complementary to the first nucleotide analogue or modified nucleotide that is identified in the cycle of the nucleic acid sequencing reaction, and wherein the second template nucleic acid comprises a nucleotide complementary to the second nucleic acid analogue or modified nucleotide.

Embodiment 53. The method of embodiment 51 or embodiment 52, wherein the first template nucleic acid and the second template nucleic acid are at optically overlapping locations in the biological sample.

Embodiment 54. The method of any one of embodiments 1-53, wherein the biological sample is a cell or tissue sample.

Embodiment 55. The method of embodiment 54, wherein the biological sample is a fresh frozen tissue section.

Embodiment 56. The method of embodiment 54, wherein the biological sample is a paraffin embedded formalin fixed (FFPE) tissue section.

Embodiment 57. The method of any one of embodiments 54-56, wherein the biological sample is immobilized on a surface.

Embodiment 58. The method of any one of embodiments 54-57, wherein the template nucleic acid molecule is sequenced in situ in the cell sample or tissue sample.

Embodiment 59. The method of any one of embodiments 54-58, wherein the cell sample comprises a layer of cells deposited on a surface.

Embodiment 60. A kit for performing a cycle of a nucleic acid sequencing reaction comprising:
a first fluorescent nucleotide analogue or modified nucleotide, and
a second nucleotide analogue or modified nucleotide in a fluorescent form; and
wherein the second nucleotide analogue or modified nucleotide is configured to be modifiable to generate a non-fluorescent form of the second nucleotide analogue or modified nucleotide.

Embodiment 61. The kit of embodiment 60, further comprising a quencher that is configured for attachment to the second nucleotide analogue or modified nucleotide.

Embodiment 62. The kit of embodiment 61, wherein the quencher is configured for attachment to the second nucleotide analogue or modified nucleotide using click chemistry.

Embodiment 63. The kit of embodiment 61 or 62, wherein the quencher is configured for attachment to the second nucleotide analogue or modified nucleotide using an azide-DBCO reaction, a tetrazine-TCO reaction, or an azide-alkyne reaction.

Embodiment 64. The kit of embodiment 60, wherein the second nucleotide analogue or modified nucleotide comprises a sulfur-containing-sensitive dye that is deactivatable by exposure to a reducing agent.

Embodiment 65. A kit for performing a cycle of a nucleic acid sequencing reaction comprising:
a first fluorescent nucleotide analogue or modified nucleotide, and
a second nucleotide analogue or modified nucleotide in a non-fluorescent form; and
wherein the second nucleotide analogue or modified nucleotide is configured to be modifiable to generate a fluorescent form of the second nucleotide analogue or modified nucleotide.

Embodiment 66. The kit of embodiment 65, wherein the second nucleotide analogue or modified nucleotide in the non-fluorescent form comprises a cleavable quencher.

Embodiment 67. The kit of embodiment 65, wherein the second nucleotide analogue or modified nucleotide in the non-fluorescent form comprises a pH-sensitive dye that is activatable by protonation.

Embodiment 68. A method, comprising:
contacting a biological sample with a composition comprising a first fluorescent nucleotide analogue or modified nucleotide and a second nucleotide analogue or modified nucleotide in a fluorescent form; and
performing a cycle of a nucleic acid sequencing reaction on a template nucleic acid in the biological sample, wherein the nucleic acid sequencing reaction comprises, sequentially:
   a) detecting a fluorescent intensity of the second nucleotide analogue or modified nucleotide, wherein after detecting the fluorescent intensity, the second nucleotide analogue or modified nucleotide is modified to generate a non-fluorescent form of the second nucleotide analogue or modified nucleotide, and
   b) detecting a fluorescent intensity of the first fluorescent nucleotide analogue or modified nucleotide.

Embodiment 69. The method of embodiment 68, wherein the non-fluorescent form of the second nucleotide analogue or modified nucleotide comprises a quencher.

Embodiment 70. The method of embodiment 69, wherein the second nucleotide analogue or modified nucleotide is modified to generate the non-fluorescent form of the second nucleotide analogue or modified nucleotide by attaching the quencher to the second nucleotide analogue or modified nucleotide.

Embodiment 71. The method of embodiment 70, wherein the quencher is attached to the second nucleotide analogue or modified nucleotide by a click chemistry attachment.

Embodiment 72. The method of any one of embodiments 68-71, wherein the second nucleotide analogue or modified nucleotide in the fluorescent form is a fluorescent nucleotide analogue.

Embodiment 73. The method of embodiment 68, wherein the second nucleotide analogue or modified nucleotide in the fluorescent form comprises a fluorophore.

Embodiment 74. The method of embodiment 68, wherein the second nucleotide analogue or modified nucleotide comprises a pH-sensitive dye.

Embodiment 75. The method of embodiment 68, wherein the second nucleotide analogue or modified nucleotide comprises a reducing agent-sensitive dye comprising a disulfide moiety, wherein the disulfide moiety is cleavable upon contact with a reducing agent, and wherein the reducing agent-sensitive dye becomes non-fluorescent when the disulfide moiety is cleaved.

Embodiment 76. The method of embodiment 68, wherein the biological sample is a cell or tissue sample.

Embodiment 77. The method of embodiment 68, wherein the biological sample is immobilized on a surface.

Embodiment 78. A method, comprising:
contacting a biological sample with a composition comprising a first fluorescent nucleotide analogue or modified nucleotide and a second nucleotide analogue or modified nucleotide, wherein the second nucleotide analogue or modified nucleotide is in a non-fluorescent form; and
performing a cycle of a nucleic acid sequencing reaction comprising a template nucleic acid in the biological sample, wherein the nucleic acid sequencing reaction comprises, sequentially: a) detecting a fluorescent intensity of the first fluorescent nucleotide analogue or modified nucleotide, wherein after detecting the first fluorescent nucleotide analogue or modified nucleotide, the second nucleotide analogue or modified nucleotide is modified to generate a fluorescent form of the second nucleotide analogue or modified nucleotide, and b) detecting a fluorescent intensity of the second nucleotide analogue or modified nucleotide.

Embodiment 79. The method of embodiment 78, wherein the non-fluorescent form of the second nucleotide analogue or modified nucleotide comprises a quencher.

Embodiment 80. The method of embodiment 79, wherein the second nucleotide analogue or modified nucleotide is modified to generate a fluorescent form of the second nucleotide analogue or modified nucleotide by cleaving the quencher.

Embodiment 81. The method of embodiment 79, wherein the quencher is linked to the second nucleotide analogue or modified nucleotide via a cleavable linker.

Embodiment 82. The method of embodiment 78, wherein the second nucleotide analogue or modified nucleotide in its fluorescent form is a fluorescent nucleotide analogue.

Embodiment 83. The method of embodiment 78, wherein the second nucleotide analogue or modified nucleotide comprises a pH-sensitive dye.

Embodiment 84. The method of embodiment 83, wherein the pH-sensitive fluorescent dye is a rhodamine derivative, wherein the pH-sensitive fluorescent dye has the following structure under acidic conditions:

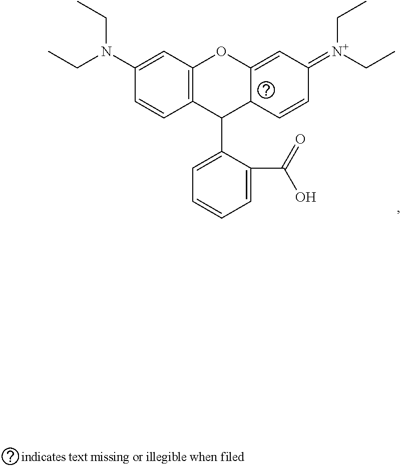 [SMILES: CCN(CC)c1ccc2c(c1)OC1=CC(=[N+](CC)CC)C=CC1C2c1ccccc1C(=O)O]

and the following structure under basic conditions:

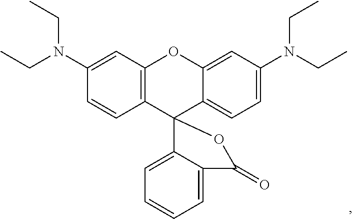 [SMILES: CCN(CC)c1ccc2c(c1)Oc1cc(N(CC)CC)ccc1C21OC(=O)c2ccccc21]

wherein the pH-sensitive dye is fluorescent in acidic conditions and non-fluorescent in basic conditions.

Embodiment 85. The method of embodiment 83, wherein an imaging buffer is used during the nucleic acid sequencing reaction, wherein the imaging buffer comprises an oxygen scavenger that releases a proton.

Embodiment 86. The method of embodiment 85, wherein the pH-sensitive dye has a pKA, and during a) the imaging buffer has a pH that is above the pKA of the pH-sensitive dye, and wherein during b) the imaging buffer has a pH that is below the pKA of the pH-sensitive dye.

Embodiment 87. The method of embodiment 78, wherein the biological sample is a cell or tissue sample.

Embodiment 88. The method of embodiment 78, wherein the biological sample is immobilized on a surface.

### EXAMPLES

The following examples are included for illustrative purposes only and are not intended to limit the scope of the present disclosure.

### Example 1: In Situ Sequencing of RCPs Using Quenchable Fluorophores

This Example provides a workflow for in situ sequencing using a multi-color system including a first nucleotide modified with a quenchable fluorophore of a higher energy color (e.g., a green dye) and a second nucleotide modified with a different fluorophore of a lower energy color (e.g., a red dye). For example, a post-incorporation conjugation reaction (e.g., a click chemistry reaction) to incorporate a fluorescence quenching dye onto a fluorophore-labeled nucleotide will inactivate the fluorophore. The use of selective fluorescence quenching permits improved sensitivity and a higher likelihood of correctly assigning a target's identity based on fluorescence. Further, the ability to inactivate fluorescence associated with individual probe targets during the detection of rolling circle products (RCPs) improves the signal, allowing for improved spatial resolution of RCPs in situ.

A biological sample (e.g., a processed or cleared biological sample, a tissue sample, a sample embedded in a hydrogel, or a fixed and paraffin embedded sample, etc.) is contacted with a probe, such as a circularizable probe or probe set (e.g., a padlock probe). The circularizable probe or probe set is hybridized to a target nucleic acid sequence in the tissue sample and is ligated to generate a closed circle (e.g., a closed unit structure) from the circularizable probe or probe set. The circular or circularized probe is then amplified by a DNA polymerase in an RCA reaction to generate a rolling circle amplification product (RCP) comprising multiple copies of a sequence complementary to the circular or circularizable probe or probe set.

Detection of target sequences in the rolling circle amplification (RCA) product (“RCP”) is performed using a base-by-base multicycle sequencing approach. During sequencing, the target nucleic acids are in a 3D sample and the RCPs are hybridized to the target nucleic acids. The RCA product is hybridized to a sequencing primer, and multiple cycles of base-by-base sequencing of nucleotides are performed. FIG. 1 depicts an exemplary workflow for a sequencing cycle that includes detecting fluorescence of two different fluorophores by sequentially: detecting a first fluorophore (a higher energy fluorophore, green in this example) associated with a first nucleotide, attaching via click chemistry a quencher to the first nucleotide thereby quenching the first fluorophore, and detecting a second fluorophore (a lower energy fluorophore, red in this example) associated with a second nucleotide while the first fluorophore is quenched. Different fluorescence (e.g., different excitation and/or emission wavelengths) is associated with different nucleotides at locations in the sample. A first modified nucleotide is labeled with a “quenchable” fluorophore and has an attachment moiety (e.g., a thiol) that can conjugate to a cognate attachment moiety on a quencher (e.g., TQ-1 conjugated to maleimide). Prior to the addition of the quencher, the first modified nucleotide is “ON” and activated (FIG. 1, left). At this stage, the first modified nucleotide is detected and imaged. After incorporating the fluorescence quencher at the attachment moiety, the quenched fluorophore is inactivated and “OFF” (FIG. 1, right). After the first modified nucleotide is quenched, a second modified nucleotide labeled with a second fluorophore (a “steady state” fluorophore in this example) is imaged and detected. The steady state fluorophore remains “ON” throughout imaging and the sequencing process.

During each cycle, the primed RCA product is contacted with the first modified nucleotide labeled with the first fluorophore and the second modified nucleotide labeled with the second fluorophore. As shown in FIG. 1, the first fluorophore is initially unquenched or “ON”. In a multi-color system, a higher energy fluorophore can interfere with a lower energy fluorophore during detection. In this Example, the first fluorophore is a higher energy fluorophore than the second fluorophore (e.g., the first fluorophore is a green fluorophore, and the second fluorophore is a red fluorophore). Thus, the sample is first imaged with excitation and emission spectra optimized for the first fluorophore. Once imaging of the first fluorophore is completed, the first modified nucleotide is further modified to incorporate a fluorescence quencher at the attachment moiety. Imaging proceeds once the first (higher energy) fluorophore is “OFF” and inactivated. The excitation and emission spectra are now optimized for the steady state second fluorophore (which is “ON” throughout the cycle), and imaging of the steady state second fluorophore occurs. This cycle is repeated, and images are obtained in each cycle. One or more wash steps are performed during each cycle or between cycles.

This Example provides a workflow for the use of chemical modifications to quench fluorescence in a multi-color system to better discriminate fluorescent signals in the same sample, including in situ. The ability to selectively inactivate a fluorophore improves imaging and optical signal-to-noise ratio in a multi-color system. This Example shows the use of quenchable fluorophores in ameliorating optical crowding when detecting multiple fluorescent signals.

### Example 2: In Situ Sequencing of RCPs with Activatable Fluorophores

This Example provides a workflow for in situ sequencing using a multi-color system including a first fluorophore of a higher energy color (e.g., green in this example) activatable by removal of a quencher, and a second fluorophore of a lower energy color than the first fluorophore (e.g., red in this example). Cleavage of a cleavable moiety (e.g., a disulfide bond) that releases a quencher will activate the fluorophore, including in a biological sample (e.g., in situ). Selectively activating fluorescence permits improved sensitivity and a higher likelihood of correctly assigning a nucleotide at a spatial location in a sample. The ability to activate fluorescence of a particular fluorophore during sequencing using a multi-color system improves the signal, allowing for improved spatial resolution of targets in a sample.

A biological sample (e.g., a processed or cleared biological sample, a tissue sample, a sample embedded in a hydrogel, or a fixed and paraffin embedded sample, etc.) is contacted with a probe (e.g., circularizable probe or probe set), which hybridizes and is further ligated as described in Example 1. The circular or circularized probe is then amplified by a DNA polymerase in an RCA reaction to generate a rolling circle amplification product (RCP) comprising multiple copies of a sequence complementary to the circular or circularizable probe or probe set.

Detection of target sequences in the rolling circle amplification (RCA) product is performed using a base-by-base multicycle sequencing approach. During sequencing, the target nucleic acids are in a 3D sample, the RCPs are hybridized to the target nucleic acids, and sequencing of the RCPs is performed using a multi-color, base-by-base sequencing assay. The RCP is hybridized to a sequencing primer, and multiple cycles of base-by-base sequencing of nucleotides are performed. FIG. 2 depicts an exemplary workflow for a sequencing cycle that includes detecting fluorescence of different fluorophores associated with RCPs by sequentially: detecting a first fluorophore (a lower energy fluorophore, red in this example) while a second fluorophore is quenched (higher energy fluorophore, green in this example), removing a quencher associated with the second fluorophore, and detecting the second fluorophore (“unquenched fluorophore”). During each cycle, the fluorescence associated with a specific nucleotide is measured. As shown in FIG. 2, initially, the quenched fluorophore is “OFF” and the sample is first imaged with excitation and emission spectra optimized for the other, steady state fluorophore. Once imaging of the steady state fluorophore is complete, the cleavage moiety releasing the quencher is cleaved. Once cleaved, the fluorophore is activated, and the sample is then imaged with excitation and emission spectra optimized for the second (unquenched) fluorophore. Fluorescent images are obtained in each cycle, and one or more wash steps are performed in a cycle or between cycles. Images at multiple spatial locations across the sample may be taken during a single imaging step. Different fluorescence (e.g., different excitation and/or emission wavelengths) is associated with different nucleotides at locations in the sample. By quenching the second fluorophore, detection of the first fluorophore is improved during the initial imaging step, as compared to imaging the first fluorophore while a second fluorophore is ON.

This Example provides a workflow for the use of chemical reactions to remove fluorescent quenchers and better discriminate fluorescent signals in the same sample, including in situ. Imaging and optical signal-to-noise ratio are improved in a multi-color system by the ability to selectively transition the fluorescence state of a fluorophore.

### Example 3: In Situ Sequencing of RCPs with pH-Sensitive Fluorophores

This Example provides a workflow for in situ sequencing using a multi-color system including a pH-sensitive fluorophore and an additional fluorophore. pH-sensitive fluorophores permit improved sensitivity and a higher likelihood of correctly assigning a nucleotide at a spatial location in a sample, based on fluorescence. The ability to modulate pH-sensitive fluorophores during sequencing using a multi-color system improves the fluorophore signal, allowing for improved spatial resolution of targets in a sample.

This Example provides a workflow for in situ sequencing using pH-sensitive fluorophores. In this example, an imaging buffer is used that decreases in pH after one round of imaging. The imaging buffer includes an oxygen scavenger that releases a proton for each molecular oxygen molecule that is consumed.

FIG. 3 depicts an exemplary workflow for a sequencing cycle that includes detecting fluorescence of a pH-sensitive fluorophore that becomes activated in an acidic environment in situ (e.g., in a 3D context). As shown in FIG. 3 (left panel), the pH-sensitive fluorophore is initially “OFF”. The pH-sensitive fluorophore is a rhodamine derivative that has a closed spirolactam ring while in the OFF state. In an initial imaging step, a first fluorophore (a “steady state” fluorophore) is imaged using excitation and emission spectra optimized for the first steady state fluorophore. The imaging takes place in an imaging buffer that includes an oxygen scavenger. During the initial imaging, the pH of the imaging buffer is at a pH higher than the pKA of the pH-sensitive fluorophore. As oxygen molecules are scavenged during this initial imaging step, protons are released, and the pH is decreased.

After imaging the steady state fluorophore, the lower pH of the imaging buffer activates the pH-sensitive fluorophore to “ON”. In its protonated form, the spirolactam ring of the fluorophore opens into an amide form activating the fluorescence of the molecule. The steady state fluorophores remain “ON” throughout imaging and the sequencing process.

The sample is then imaged with excitation and emission spectra optimized for the pH-sensitive fluorophore. A wash step is performed prior to repeating the sequencing cycle. As previously described in Example 1, multiple cycles of contacting the sample with probes and sequence determination are performed

This Example provides a workflow for the use of varying pH in an environment to modulate the fluorescence of pH-sensitive fluorophores. Imaging and optical signal-to-noise ratio are improved in a multi-color system by the ability to selectively transition the fluorescence state of a fluorophore in the same sample, including in situ.

### Example 4: In Situ Sequencing of RCPs with Reducing Agent-Sensitive Fluorophores

This Example provides a workflow for in situ sequencing using a multi-color system including a reducing agent-sensitive fluorophore of a higher energy color (e.g., green in this example) and an additional fluorophore of a color that is lower energy than the reducing agent-sensitive fluorophore (e.g., red in this example). Reducing agent-sensitive fluorophores permit improved sensitivity and a higher likelihood of correctly assigning a nucleotide at a spatial location in a sample, based on fluorescence. In a multi-color in situ sequencing system, the ability to modulate reducing agent-sensitive fluorophores improves detection of other fluorophores in the system and thus improves spatial resolution of targets in a sample.

This Example provides a workflow for in situ sequencing using a first nucleotide attached to a first fluorophore that is a reducing agent-sensitive fluorophore, and a second nucleotide attached to a second fluorophore, a third nucleotide attached to a third fluorophore, and a fourth nucleotide attached to a fourth fluorophore.

FIG. 4 depicts an exemplary workflow for a sequencing cycle that includes detecting fluorescence of the first nucleotide, adding a reducing agent to deactivate fluorescent of the first nucleotide, and then detecting the second nucleotide. As shown in FIG. 4 (left panel), the reducing agent-sensitive fluorophore is initially “ON”. The reducing agent-sensitive fluorophore is a sulfo-Cy5 derivative dye, modified with a moiety including a disulfide bond. Once exposed to a reducing agent (e.g., TCEP), the disulfide is converted to a thiol, which disrupts the resonance structure of the dye and blocks the fluorescence. In a first imaging step, the reducing agent-sensitive fluorophore is imaged using an excitation wavelength optimized for the reducing agent-sensitive fluorophore. The imaging takes place in an imaging buffer without a reducing agent.

After the first imaging step, a reducing agent is added to disrupt the disulfide bond and generate a free thiol, which blocks fluorescents of the reducing agent-sensitive fluorophore. Next, a second imaging step is performed to detect the second nucleotide, using an excitation wavelength optimized for the fluorophore of the second nucleotide.

The sample is then imaged with excitation and emission spectra optimized for the pH-sensitive fluorophore. A wash step is performed prior to repeating the sequencing cycle. As previously described in Example 1, multiple cycles of contacting the sample with probes and sequence determination are performed

This Example provides a workflow for the use of a reducing agent to modulate the fluorescence of a reducing agent-sensitive fluorophore. Imaging and optical signal-to-noise ratio are improved in a multi-color system by the ability to selectively transition the fluorescence state of a fluorophore.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

What is claimed is:

1. A method, comprising:
contacting a biological sample with a composition comprising a first fluorescent nucleotide analogue or modified nucleotide and a second nucleotide analogue or modified nucleotide in a fluorescent form; and
performing a cycle of a nucleic acid sequencing reaction on a template nucleic acid in the biological sample, wherein the nucleic acid sequencing reaction comprises, sequentially:
a) detecting a fluorescent intensity of the second nucleotide analogue or modified nucleotide, wherein after detecting the fluorescent intensity, the second nucleotide analogue or modified nucleotide is modified to generate a non-fluorescent form of the second nucleotide analogue or modified nucleotide, and
b) detecting a fluorescent intensity of the first fluorescent nucleotide analogue or modified nucleotide.

2. The method of claim 1, wherein the non-fluorescent form of the second nucleotide analogue or modified nucleotide comprises a quencher.

3. The method of claim 2, wherein the second nucleotide analogue or modified nucleotide is modified to generate the non-fluorescent form of the second nucleotide analogue or modified nucleotide by attaching the quencher to the second nucleotide analogue or modified nucleotide.

4. The method of claim 3, wherein the quencher is attached to the second nucleotide analogue or modified nucleotide by a click chemistry attachment.

5. The method of claim 1, wherein the second nucleotide analogue or modified nucleotide in the fluorescent form is a fluorescent nucleotide analogue.

6. The method of claim 1, wherein the second nucleotide analogue or modified nucleotide in the fluorescent form comprises a fluorophore.

7. The method of claim 1, wherein the second nucleotide analogue or modified nucleotide comprises a pH-sensitive dye.

8. The method of claim 1, wherein the second nucleotide analogue or modified nucleotide comprises a reducing agent-sensitive dye comprising a disulfide moiety, wherein the disulfide moiety is cleavable upon contact with a reducing agent, and wherein the reducing agent-sensitive dye becomes non-fluorescent when the disulfide moiety is cleaved.

9. The method of claim 1, wherein the biological sample is a cell or tissue sample.

10. The method of claim 1, wherein the biological sample is immobilized on a surface.

11. A method, comprising:
contacting a biological sample with a composition comprising a first fluorescent nucleotide analogue or modified nucleotide and a second nucleotide analogue or modified nucleotide, wherein the second nucleotide analogue or modified nucleotide is in a non-fluorescent form; and
performing a cycle of a nucleic acid sequencing reaction comprising a template nucleic acid in the biological sample, wherein the nucleic acid sequencing reaction comprises, sequentially: a) detecting a fluorescent intensity of the first fluorescent nucleotide analogue or modified nucleotide, wherein after detecting the first fluorescent nucleotide analogue or modified nucleotide, the second nucleotide analogue or modified nucleotide is modified to generate a fluorescent form of the second nucleotide analogue or modified nucleotide, and b) detecting a fluorescent intensity of the second nucleotide analogue or modified nucleotide.

12. The method of claim 11, wherein the non-fluorescent form of the second nucleotide analogue or modified nucleotide comprises a quencher.

13. The method of claim 12, wherein the second nucleotide analogue or modified nucleotide is modified to generate a fluorescent form of the second nucleotide analogue or modified nucleotide by cleaving the quencher.

14. The method of claim 12, wherein the quencher is linked to the second nucleotide analogue or modified nucleotide via a cleavable linker.

15. The method of claim 11, wherein the second nucleotide analogue or modified nucleotide in its fluorescent form is a fluorescent nucleotide analogue.

16. The method of claim 11, wherein the second nucleotide analogue or modified nucleotide comprises a pH-sensitive dye.

17. The method of claim 16, wherein the pH-sensitive fluorescent dye is a rhodamine derivative, wherein the pH-sensitive fluorescent dye has the following structure under acidic conditions: 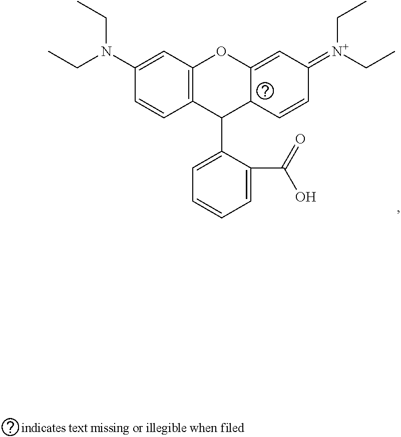 [SMILES: CCN(CC)c1ccc2c(c1)OC1=CC(=[N+](CC)CC)C=CC1C2c1ccccc1C(=O)O]
and the following structure under basic conditions: 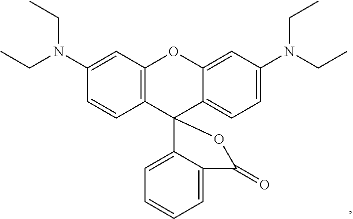 [SMILES: CCN(CC)c1ccc2c(c1)Oc1cc(N(CC)CC)ccc1C21OC(=O)c2ccccc21]
wherein the pH-sensitive dye is fluorescent in acidic conditions and non-fluorescent in basic conditions.

18. The method of claim 16, wherein an imaging buffer is used during the nucleic acid sequencing reaction, wherein the imaging buffer comprises an oxygen scavenger that releases a proton.

19. The method of claim 18, wherein the pH-sensitive dye has a pKA, and during the detecting in a) the imaging buffer has a pH that is above the pKA of the pH-sensitive dye, and wherein during the detecting in b) the imaging buffer has a pH that is below the pKA of the pH-sensitive dye.

20. The method of claim 11, wherein the biological sample is a cell or tissue sample.

21. The method of claim 11, wherein the biological sample is immobilized on a surface.
